(19) United States
(12) Patent Application Publication
(10) Pub. No.: US 2016/0262868 A1
(43) Pub. Date: Sep. 15, 2016

(54) GRAFT DEVICES WITH SPINES AND RELATED SYSTEMS AND METHODS

(71) Applicant: NEOGRAFT TECHNOLOGIES, INC., Taunton, MA (US)

(72) Inventors: Lorenzo SOLETTI, Pittsburgh, PA (US); John SPIRIDIGLIOZZI, Boston, MA (US); Mohammed S. El-KURDI, Mansfield, MA (US); Jon MCGRATH, Duxbury, MA (US); J. Christopher FLAHERTY, Topsfield, MA (US)

(73) Assignee: NEOGRAFT TECHNOLOGIES, INC., Taunton, MA (US)

(21) Appl. No.: 15/023,265

(22) PCT Filed: Sep. 18, 2014
(86) PCT No.: PCT/US2014/056371

§ 371 Date: Mar. 18, 2016

Related U.S. Application Data
(60) Provisional application No. 61/880,550, filed on Sep. 20, 2013.

(51) Int. Cl. A61F 2/07

(52) U.S. Cl. CPC A61F 2/07; A61F 2002/072

(57) ABSTRACT

A graft device for a mammalian patient tubular conduit comprises a fiber matrix surrounding the tubular conduit and a spine comprising a first support portion and a second support portion. At least one of the first support portion or the second support portions can be constructed and arranged to rotate relative to the other to receive the tubular conduit. The first support portion can comprise a first set of projections and the second support portion can comprise a second set of projections that interdigitiate with the first set of projections. Tools for creating the spine and for applying the spine are also provided.

### TECHNICAL FIELD

This disclosure relates generally to graft devices for a mammalian patient, and more particularly graft devices which include a spine, and to related systems and methods.

### BACKGROUND

Coronary artery disease, leading to myocardial infarction and ischemia, is currently a leading cause of morbidity and mortality worldwide. Current treatment alternatives consist of percutaneous transluminal angioplasty, stenting, and coronary artery bypass grafting (CABG). CABG can be carried out using either arterial or venous conduits and is an effective and widely used treatment to combat coronary arterial stenosis, with nearly 500,000 procedures being performed annually. In addition, there are approximately 80,000 lower extremity bypass surgeries performed annually. The venous conduit used for bypass procedures is most frequently the autogenous saphenous vein and remains the graft of choice for 95% of surgeons performing these bypass procedures. According to the American Heart Association, in 2004 there were 427,000 bypass procedures performed in 249,000 patients. The long term outcome of these procedures is limited due to occlusion of the graft vein or anastomotic site as a result of intimal hyperplasia (IH), which can occur over a timeframe of months to years.

Development of successful small diameter synthetic or tissue engineered vascular grafts has yet to be accomplished and use of arterial grafts (internal mammary, radial, or gastroepiploic arteries, for example) is limited by the short size, small diameter and availability of these veins. Despite their wide use, failure of arterial vein grafts (AVGs) remains a major problem: 12% to 27% of AVGs become occluded in the first year with a subsequent annual occlusive rate of 2% to 4%. Patients with failed AVGs usually require clinical intervention such as an additional surgery.

IH accounts for 20% to 40% of all AVG failures within the first 5 years after CABG surgery. Several studies have determined that IH develops, to some extent, in all mature AVGs and this development is regarded by many as an unavoidable response of the vein to grafting. IH is characterized by phenotypic modulation, followed by de-adhesion and migration of medial and adventitial smooth muscle cells (SMCs) and myofibroblasts into the intima where they proliferate. In many cases, this response can lead to stenosis and diminished blood flow through the graft. It is thought that IH may be initiated by the abrupt exposure of the veins to the dynamic mechanical environment of the arterial circulation.

For these and other reasons, there is a need for systems, methods and devices which provide enhanced AVGs and other improved grafts for mammalian patients. Desirably, the systems, methods and devices will improve long term patency and minimize surgical and device complications such as those caused by kinking of graft devices.

### SUMMARY

Embodiments of the present inventive concepts described herein can be directed to graft devices for mammalian patients, as well as systems and methods for producing these graft devices.

According to one aspect of the technology described herein, a graft device for a mammalian patient comprises a tubular conduit; a fiber matrix surrounding the tubular conduit; and a spine. In some embodiments, the spine can comprise a first support portion and a second support portion, and at least one of the first support portion or the second support portions is constructed and arranged to rotate relative to the other to receive the tubular conduit. Alternatively or additionally, the spine can comprise a first support portion comprising a first set of projections, and a second support portion comprising a second set of projections, and the first set of projections can interdigitate with the second set of projections.

In some embodiments, the spine is constructed and arranged to provide one or more functions selected from the group consisting of: minimizing undesirable conditions, such as buckling, conduit deformation, luminal deformation, stasis, flows characterized by significant secondary components of velocity vectors such as vortical, recirculating or turbulent flows, luminal collapse, and/or thrombus formation; preserving laminar flow such as preserving laminar flow with minimal secondary components of velocity, such as blood flow through the graft device, blood flow proximal to the graft device and/or blood flow distal to the graft device; preventing bending and/or allowing proper bending of the graft device, such as bending that occurs during and/or after the implantation procedure; preventing accumulation of debris; preventing stress concentration on the tubular wall; maintaining a defined geometry in the tubular conduit: preventing axial rotation about the length of the tubular conduit; and combinations thereof.

In some embodiments, the spine is positioned between the tubular conduit and the fiber matrix.

In some embodiments, the fiber matrix comprises an outer surface and the spine is positioned on the fiber matrix outer surface.

In some embodiments, the fiber matrix comprises a thickness between 100 microns and 1000 microns, such as a thickness between 1450 microns and 400 microns, or a thickness of approximately 250 microns.

In some embodiments, the fiber matrix comprises an inner layer and an outer layer, and the spine is positioned between the fiber matrix inner layer and outer layer. The fiber matrix can comprise a first thickness and the inner layer can comprise a second thickness approximately between 1% and 99% of the first thickness, such as between 25% and 60% of the first thickness, such as approximately 33% of the first thickness. The inner layer can comprise a layer of between approximately 62 microns and 83 microns in thickness.

In some embodiments, the fiber matrix comprises a first elastic modulus and the spine comprises a second elastic modulus similar to the first elastic modulus.

In some embodiments, the spine is constructed and arranged to be positioned in the graft device prior to application of the fiber matrix to the graft device.

In some embodiments, the spine is constructed and arranged to be positioned in the graft device during application of the fiber matrix to the graft device.

In some embodiments, the spine is constructed and arranged to be positioned on the graft device after application of the fiber matrix to the graft device.

In some embodiments, the spine is constructed and arranged to be applied to the tubular conduit with a tool.

In some embodiments, the spine is resiliently biased in a tubular shape. The spine can be resiliently biased to apply a radial outward force to an anastomotic site.

In some embodiments, the spine comprises at least a malleable portion.

In some embodiments, the spine comprises multiple interdigitating projections. The first support portion can comprise a first set of projections and the second support portion can comprise a second set of projections that alternatively interdigitate with the first set of projections. The interdigitating projections can overlap, such as an overlap of at least 1.0 mm, 1.1 mm or 1.4 mm. The multiple interdigitating projections can comprise multiple loops of a filament. The multiple loops can each comprise a tip diameter between 0.020″ and 0.064″, such as a tip diameter of approximate 0.042″. The multiple interdigitating projections can comprise at least three interdigitating projections, such as at least 6 interdigitating projections. The spine can comprise at least two interdigitating projections for each 15 mm of length, or for each 7.5 mm of length, or for each 2 mm of length. The spine can comprise approximately two interdigitating projections for each 6.5 mm of length.

In some embodiments, the spine is constructed and arranged to be cut to a determined length.

In some embodiments, the first support portion comprises a first elongate member with a relatively continuous partial circumferential cross section and the second support portion comprises a second elongate member with a relatively continuous partial circumferential cross section. The first elongate member partial circumferential cross section can span an arc of 180° or less. The first elongate member and second elongate member can be operably attached.

In some embodiments, the spine comprises at least one continuous filament. The at least one continuous filament comprises a continuous length of at least 65 inches, such as a length of at least 75 inches or at least 85 inches. The spine can comprise a length of 3.5 inches when the at least one continuous filament comprises a continuous length of at least 85 inches. The at least one continuous filament comprises an extruded filament. The at least one continuous filament can comprise a molded filament. The at least one continuous filament can comprise at least a portion with a cross sectional geometry selected from the group consisting of: elliptical; circular; oval; square; star; spiral-shaped; rectangular; trapezoidal; parallelogram-shaped; rhomboid-shaped; T-shaped; and combinations thereof. The at least one continuous filament comprises at least a portion with a relatively circular cross section. The at least one continuous filament can comprise at least a portion with a cross section with a major axis less than or equal to 0.8 mm, such as when the at least one continuous filament comprises a relatively circular cross section and/or a relatively oval cross section. The at least one continuous filament can comprise a cross section with a major axis between 0.2 mm and 1.5 mm. The at least one continuous filament can comprise a cross section with a major axis less than or equal to 1.5 mm. The at least one continuous filament can comprise a cross section with a major axis less than or equal to 0.8 mm, such as less than or equal to 0.6 mm, or a cross section between 0.4 mm and 0.5 mm. The at least one continuous filament can comprise a cross section with a major axis greater than or equal to 0.1 mm, such as greater than or equal to 0.3 mm. The at least one continuous filament can comprise a monofilament structure. The at least one continuous filament can comprise a multiple filament structure, such as a braided structure.

In some embodiments, the spine comprises an injection molded component.

In some embodiments, the spine comprises a thermoset plastic component, such as when the spine comprises multiple thermoset interdigitating projections.

In some embodiments, the spine comprises an electrospun component, such as when the fiber matrix comprises an electrospun component. The spine and fiber matrix can comprise similar materials.

In some embodiments, the spine comprises a surface and at least a portion of the surface comprises a modified surface. The modified surface can comprise a surface modified with a solvent. The surface can be modified with a solvent selected from the group consisting of: dimethylformamide; hexafluoroisopropanol; tetrahydrofuran; dimethyl sulfoxide; isopropyl alcohol; ethanol; and combinations thereof. The modified surface can comprise a surface modified to enhance adhesion of the spine to at least one of the tubular conduit or the fiber matrix. The modified surface can comprise a surface with a modified surface energy. The modified surface can comprise a surface modified with a heated die comprising a textured surface. The modified surface can comprise a surface modified with radiofrequency plasma glow discharge. The modified surface can comprise a surface modified with radiofrequency plasma glow discharge performed in the presence of a material selected from the group consisting of: hydrogen; nitrogen; ammonia; oxygen; carbon dioxide; C₂F₆; C₂F₄; C₃F₆; C₂H₄; C_{Z}H_{Z}, CH₄; and combinations thereof.

In some embodiments, the spine comprises a relatively tubular structure. The tubular structure can comprise an internal diameter along its length and the tubular conduit can comprise an outer diameter along its length, and the spine inner diameter can approximate the tubular conduit outer diameter. The tubular structure can comprise an inner diameter of at least 2 mm. The tubular structure can comprise an inner diameter of less than or equal to 20 mm. The tubular structure can comprise a length between 2 inches and 6 inches, such as a length between 3 inches and 5 inches. The spine can comprise multiple tubular structures, such as when one or more tubular structures comprise a length between 1 inches and 6 inches.

In some embodiments, the spine comprises an inner diameter of approximately 4.0 mm and the tubular conduit comprises a diameter between approximately 3.57 mm and 4.37 mm.

In some embodiments, the spine comprises an inner diameter of approximately 4.7 mm and the tubular conduit comprises a diameter between approximately 4.37 mm and 5.27 mm.

In some embodiments, the spine comprises an inner diameter of approximately 5.50 mm and the tubular conduit comprises a diameter between approximately 5.27 mm and 6.17 mm.

In some embodiments, the spine is constructed and arranged to laterally attach to the tubular conduit.

In some embodiments, the spine and the fiber matrix comprise similar materials. The spine can comprise an extruded component and the fiber matrix comprises an electrospun component.

In some embodiments, the spine comprises at least one thermoplastic co-polymer. The spine can comprise a first material and a second different material. The second material can comprise a softer material than the first material. The spine can comprise relatively equal amounts of the first material and the second material. The softer, second material can comprise polydimethylsiloxane and a polyether-based polyurethane. The harder, first material can comprise aromatic methylene diphenyl isocyanate.

In some embodiments, the spine comprises a material with a durometer of approximately 55 D.

In some embodiments, spine comprises a material with a durometer of between 52 D and 120 R, such as a durometer between 52 D and 85 D, or between 52 D and 62 D.

In some embodiments, the spine comprises a polymer. The spine can comprise a polymer selected from the group consisting of: silicone; polyether block amide; polypropylene; nylon; polytetrafluoroethylene; polyethylene; ultra high molecular weight polyethylene; polycarbonates; polyolefins; polyurethanes; polyvinylchlorides; polyamides; polyimides; polyacrylates; polyphenolics; polystyrene; polycaprolactone; polylactic acid; polyglycolic acid; polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations thereof.

In some embodiments, the spine comprises a resin reinforced with at least one of carbon fiber or Kevlar.

In some embodiments, the spine comprises a metal. The spine can comprise a metal selected from the group consisting of: nickel titanium alloy; titanium alloy; titanium; stainless steel; tantalum; magnesium; cobalt-chromium alloy; gold; platinum; and combinations thereof.

In some embodiments, the spine comprises a biodegradable material. In some cases, the fiber matrix can comprise a biodegradable material and/or a non-biodegradable material.

In some embodiments, the spine comprises a non-biodegradable material. In some cases, the fiber matrix can comprise a biodegradable material and/or a non-biodegradable material.

In some embodiments, the spine comprises a biodegradable material and a non-biodegradable material. The spine can comprise a material constructed and arranged to remain relatively intact or otherwise unchanged for at least 6 months. In some cases, the fiber matrix can comprise a biodegradable material and/or a non-biodegradable material.

In some embodiments, the spine comprises a polymer constructed and arranged to change one or more properties upon exposure to an external stimuli. The polymer can comprise a polymer selected from the group consisting of: N-isopropylacrylamide (NIPAAm); a polaxamer (Pluronics); and combinations thereof. The external stimuli can comprise a stimuli selected from the group consisting of: temperature; pH; light; magnetic field; electric field; exposure to a solvent; and combinations thereof. The property changed can comprise a property selected from the group consisting of: hydrophobicity; a material property, an adhesive property; size; geometry; and combinations thereof. The hydrophobicity can increase upon exposure to the external stimuli. The external stimuli can comprise an electromagnetic field. The electromagnetic field can comprise an electromagnetic field used during an electrospinning process.

In some embodiments, at least a portion of the spine is radiopaque.

In some embodiments, the spine comprises a coating. The spine can comprise at least one continuous filament with an external surface and the coating can be positioned on at least a portion of the filament external surface. The spine can comprise an inner surface and the coating can be positioned on the filament inner surface. The spine can comprise an outer surface and the coating can be positioned on the filament outer surface. The coating can be constructed and arranged to provide a function selected from the group consisting of: anti-thrombogenecity; anti-proliferation; anti-calcification; vasorelaxation; and combinations thereof. The coating can comprise a dehydrated gelatin. The dehydrated gelatin can be constructed and arranged to hydrate and adhere to the tubular conduit. The coating can comprise a coating with adhesive properties. The coating can comprise a material selected from the group consisting of: fibrin gel; starch-based compound; mussel adhesive proteins; hydrophilic coating; hydrophobic coating; radiopaque coating; and combinations thereof.

In some embodiments, the spine comprises an adhesive element constructed and arranged to adhere the spine to at least one of the tubular conduit or the fiber matrix. The adhesive element can comprise a coating.

In some embodiments, the device further comprises one or more fasteners constructed and arranged to apply a retention force between at least two of: the tubular conduit, the spine or the fiber matrix. The one or more fasteners can be attached to the spine. The one or more fasteners can comprise one or more elements selected from the group consisting of: adhesive; staple; clip; suture; barb; hook; and combinations thereof. The one or more fasteners can comprise at least four fasteners. The tubular conduit can be placed on a mandrel and the one or more fasteners can be applied to the tubular conduit when the mandrel is positioned within the tubular conduit. The graft device includes a proximal end and a distal end and at least one fastener is placed within 1 cm of the proximal end and at least one fastener is placed within 1 cm of the distal end. The spine can comprise multiple interdigitating projections, and the one or more fasteners and the multiple interdigitating projections can comprise a similar material.

In some embodiments, the device further comprises a kink-resisting element. The spine can comprise the kink-resisting element.

In some embodiments, the tubular conduit comprises a varying circumferential shape and the fiber matrix conforms to the varying circumferential shape of the tubular conduit.

In some embodiments, the tubular conduit comprises harvested tissue. The tubular conduit can comprise a harvested vessel such as a harvested vein. The harvested tissue can comprise tissue selected from the group consisting of: saphenous vein; vein; artery; urethra; intestine; esophagus; ureter; trachea; bronchi; duct; fallopian tube; and combinations thereof.

In some embodiments, the tubular conduit comprises an artificial material. The artificial material can comprise a material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester-based polymer; polyether-based polymer; thermoplastic rubber; and combinations thereof.

In some embodiments, the fiber matrix comprises at least one polymer. The fiber matrix can comprise a polymer selected from the group consisting of; polyolefins; polyurethanes; polyvinylchlorides; polyamides; polyimides; polyacrylates; polyphenolics; polystyrene; polycaprolactone; polylactic acid; polyglycolic acid; and combinations thereof. The fiber matrix can comprise a polymer applied in combination with a solvent where the solvent is selected from the group consisting of: hexafluoroisopropanol; acetone; methyl ethyl ketone; benzene; toluene; xylene; dimethyleformamide; dimethylacetamide; propanol; ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations thereof.

In some embodiments, the fiber matrix comprises a thermoplastic co-polymer comprising two or more materials. The two or more materials can comprise a first material and a second softer material.

In some embodiments, the fiber matrix comprises a non-biodegradable material.

In some embodiments, the fiber matrix comprises a biodegradable material.

In some embodiments, the fiber matrix comprises a non-biodegradable material and a biodegradable material.

In some embodiments, the fiber matrix comprises an electrospun fiber matrix.

In some embodiments, the fiber matrix comprises fibers with an average diameter between 1.0 μm and 20 μm, such as fibers with an average diameter between 5 μm and 15 μm, or between 6 μm and 12 μm.

In some embodiments, the fiber matrix comprises a fiber matrix applied with a device selected from the group consisting of: an electrospinning device; a melt-spinning device; a melt-electrospinning device; a misting assembly; a sprayer; an electrosprayer; a three-dimensional printer; and combinations thereof.

In some embodiments, the fiber matrix comprises a fiber matrix applied with a three-dimensional printer.

According to another aspect of the technology, a system for producing a graft device for a mammalian patient comprises a tubular conduit; a first spine; and a fiber matrix delivery assembly constructed and arranged to deliver a fiber matrix to surround the tubular conduit.

In some embodiments, the system is constructed and arranged to manufacture a graft device as described hereabove.

In some embodiments, the first spine comprises at least one spine with an approximate inner diameter of 4.0 mm, 4.7 mm or 5.5 mm.

In some embodiments, the system further comprises a second spine. The first spine can comprise a first inner diameter and the second spine can comprise a second inner diameter different than the first inner diameter. The first inner diameter and the second inner diameter can comprise approximate diameters selected from the group consisting of: 4.0 mm; 4.7 mm and 5.5 mm. The system can further comprise a third spine. The first spine can comprise a first inner diameter, the second spine can comprise a second inner diameter different than the first inner diameter, and the third spine can comprise a third inner diameter different than the first inner diameter and the second inner diameter. The first inner diameter can comprise a diameter of approximately 4.0 mm, the second inner diameter can comprise a diameter of approximately 4.7 mm and the third inner diameter can comprise a diameter of approximately 5.5 mm.

In some embodiments, the spine comprises an inner diameter of approximately 4.0 mm and the tubular conduit comprises a diameter between approximately 3.57 mm and 4.37 mm.

In some embodiments, the spine comprises an inner diameter of approximately 4.7 mm and the tubular conduit comprises a diameter between approximately 4.37 mm and 5.27 mm.

In some embodiments, the spine comprises an inner diameter of approximately 5.50 mm and the tubular conduit comprises a diameter between approximately 5.27 mm and 6.17 mm.

In some embodiments, the system further comprises a polymer, and the polymer is provided to the fiber matrix delivery assembly to deliver the fiber matrix to surround the tubular conduit. The polymer can comprise a polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations thereof. The system can further comprise a solvent provided with the polymer to the fiber matrix delivery assembly, and the solvent can be selected from the group consisting of: hexafluoroisopropanol; acetone; methyl ethyl ketone; benzene; toluene; xylene; dimethyleformamide; dimethylacetamide; propanol; ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations thereof. The polymer can comprise a first material and a softer second material.

In some embodiments, the system further comprises a spine application tool constructed and arranged to apply the spine about the tubular conduit. The spine application tool can be constructed and arranged to laterally apply the spine about the tubular conduit. The fiber matrix can comprise an inner layer and an outer layer, and the spine application tool can be constructed and arranged to apply the spine between the inner and outer layer of the fiber matrix. The spine application tool can comprise an automated tool. The spine application tool can comprise a robotic tool. The fiber matrix delivery assembly can comprise the spine application tool. The spine application tool can comprise a scissor-like construction.

In some embodiments, the system further comprises a trimming tool constructed and arranged to trim one or both ends of the spine. The trimming tool can be further constructed and arranged to trim the fiber matrix. The trimming tool can comprise a tool selected from the group consisting of: scissors; scalpel; laser cutter; radiofrequency cutter; and combinations thereof. The fiber matrix delivery assembly can comprise the trimming tool. The trimming tool comprises an automated tool. The trimming tool comprises a robotic tool. The fiber matrix delivery assembly can comprise the trimming tool. The trimming tool can comprise a laser.

In some embodiments, the system further comprises a surface modifying agent.

In some embodiments, the system further comprises a spine fabrication tool constructed and arranged to produce the spine. The spine fabrication tool can comprise a rod and multiple pins. The rod can comprise a relatively linear rod. The rod can comprise at least a non-linear portion. The fiber matrix delivery assembly can comprise the spine fabrication tool. The fiber matrix delivery assembly can comprise an electrospinning device constructed and arranged to produce the spine. The fiber matrix delivery assembly can comprise a three-dimensional printer constructed and arranged to produce the spine. The fiber matrix delivery assembly can comprise a stereolithography device constructed and arranged to produce the spine. The fiber matrix delivery assembly comprises a fuse deposition device constructed and arranged to produce the spine.

In some embodiments, the fiber matrix delivery assembly comprises a device selected from the group consisting of: an electrospinning device; a melt-spinning device; a melt-electrospinning device; a misting assembly; a sprayer; an electrosprayer; a three-dimensional printer; and combinations thereof.

In some embodiments, the fiber matrix delivery assembly comprises an electrospinning device.

In some embodiments, the fiber matrix delivery assembly comprises a three-dimensional printer.

In some embodiments, the tubular conduit comprises a varying circumferential shape and the fiber matrix can conform to the varying circumferential shape of the tubular conduit.

In some embodiments, the tubular conduit comprises harvested tissue. The tubular conduit can comprise a harvested vessel, such as a harvested vein. The harvested tissue can comprise tissue selected from the group consisting of: saphenous vein; vein; artery; urethra; intestine; esophagus; ureter; trachea; bronchi; duct; fallopian tube; and combinations thereof.

In some embodiments, the tubular conduit comprises an artificial material. The artificial material can comprise a material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester based-polymer; polyether-based polymer; thermoplastic rubber; and combinations thereof.

According to another aspect of the technology, a method of producing a graft device for a patient comprises harvesting a tubular conduit from the patient; applying a spine over the tubular conduit; and applying a fiber matrix over the spine and the tubular conduit. The spine comprises a first support portion and a second support portion, and the first and second support portions are constructed and arranged to rotate relative to each other to receive the tubular conduit.

In some embodiments, the spine is manually applied to the tubular conduit.

In some embodiments, the spine is applied to the tubular conduit using a spine application tool. The spine application tool can rotate at least one of the spine first support portion or spine second support portion.

In some embodiments, the spine is applied to the tubular conduit by placing the spine laterally over the tubular conduit.

In some embodiments, the spine is applied to the tubular conduit by placing the spine axially over the tubular conduit.

In some embodiments, the method further comprises sterilizing the spine. The spine can be sterilized prior to applying the spine to the tubular conduit.

In some embodiments, the method further comprises trimming the spine. The method can further comprise trimming the fiber matrix.

In some embodiments, the method further comprises resiliently biasing the spine. The spine can be biased in a relatively linear shape. At least a portion of the spine can be biased in a relatively non-linear shape. The biasing of the spine can be performed prior to the applying of the spine to the tubular conduit. The forming tool can comprise a thermoset tool. The forming tool can bias the spine to a configuration comprising multiple interdigitating projections.

In some embodiments, the method further comprises applying one or more fasteners to the graft device.

In some embodiments, the tubular conduit comprises a varying circumferential shape and wherein the fiber matrix conforms to the varying circumferential shape of the tubular conduit.

In some embodiments, the tubular conduit comprises harvested tissue, such as a harvested vein. The harvested tissue can comprise tissue selected from the group consisting of: saphenous vein; artery; urethra; intestine; esophagus; ureter; trachea; bronchi; duct; fallopian tube; and combinations thereof.

According to an aspect of the technology, a fabrication tool for producing a spine comprises a first rod for receiving a filament, and the tool is constructed and arranged to fabricate a spine comprising the filament resiliently biased in a first geometry comprising multiple interdigitating projections.

In some embodiments, the fabrication tool is constructed and arranged to produce a spine as described hereabove.

In some embodiments, the first rod comprises a metal rod.

In some embodiments, the first rod comprises a relatively linear rod.

In some embodiments, the first rod comprises at least a portion that is curved. The fabrication tool can be constructed and arranged to produce a spine with at least a curved portion.

In some embodiments, the fabrication tool further comprises multiple pins extending radially from the first rod. The multiple pins can be constructed and arranged to position the filament in a first geometry including the multiple interdigitating projections. The multiple pins can comprise a first set of pins that lie in a first plane, and a second set of pins that lie in a second plane different than the first plane. The first rod can comprise multiple holes constructed and arranged to slidingly receive the multiple pins.

In some embodiments, the fabrication tool can further comprise a heater constructed and arranged to apply heat to the filament to resiliently bias the filament in the first geometry comprising multiple interdigitating projections.

In some embodiments, the fabrication tool further comprises an agent delivery assembly constructed and arranged to apply at least one agent to the filament to resiliently bias the filament in the first geometry comprising multiple interdigitating projections. The at least one agent can comprise a solvent.

In some embodiments, the tool can be constructed and arranged to expose the filament to a shape-forming process causing the filament to be resiliently biased in the first geometry comprising multiple interdigitating projections. The shape-forming process can comprise exposure to an agent such as a solvent or other agent. The shape-forming process can comprise exposure to an elevated temperature.

In some embodiments, the fabrication tool further comprises a second rod for receiving a filament, and the second rod can be constructed and arranged to cause the filament to be resiliently biased in a second geometry comprising multiple interdigitating projections. The first rod can comprise a first diameter and the second rod can comprise a second diameter different than the first diameter. The first geometry and the second geometry can comprise different diameters.

According to another aspect of the technology, an application tool for applying a spine to a tubular conduit comprises a force applying element constructed and arranged to apply a force to at least a first portion of a spine and an actuator assembly configured to allow an operator to cause rotation of at least a second portion of the spine.

In some embodiments, the application tool is constructed and arranged to laterally apply the spine to the tubular conduit.

In some embodiments, the application tool is constructed and arranged to at least one of: laterally or axially apply the spine to the tubular conduit.

In some embodiments, the spine comprises a first support portion and a second support portion, and the application tool is constructed and arranged to rotate the first portion relative to the second portion to create an opening that receives the tubular conduit.

In some embodiments, the application tool comprises a scissor-like construction.

In some embodiments, the actuator assembly is constructed and arranged to transition from an unactivated state to an activated state. The force applying element can be constructed an arranged to apply the force on the at least a first portion of the spine when the actuator assembly is in the activated state. The force applying element can be constructed and arranged to transition from a compact state to an expanded state when the actuator assembly transitions from the unactivated state to the activated state.

In some embodiments, the force applying element comprises two elongate members, and the actuator is constructed and arranged to cause the two elongate members to translate away from each other when the actuator transitions from an unactivated state to an activated state. The two elongate members can comprise two members selected from the group consisting of: tubes; plates; rods; and combinations thereof. The force applying element can be constructed and arranged to be at least partially surrounded by spine when the actuator is in an unactivated state. The application tool can be constructed and arranged to maintain the geometry of the spine prior to application.

According to another aspect of the technology, a system for producing a graft device for a mammalian patient comprises a tubular conduit and a fiber matrix delivery assembly constructed and arranged to deliver a fiber matrix to surround the tubular conduit. The fiber matrix delivery assembly comprises a three-dimensional printer.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 is a perspective, partial cut-away view of a graft device including a spine, consistent with the present inventive concepts.

FIG. 1A is a perspective view of the spine of the graft device of FIG. 1, consistent with the present inventive concepts.

FIG. 1B is a perspective view of the graft device of FIG. 1, prior to application of at least an outer layer of a fiber matrix, consistent with the present inventive concepts.

FIG. 2A is an end sectional view of a graft device including a spine placed between a tubular conduit and a fiber matrix, consistent with the present inventive concepts.

FIG. 2B is an end sectional view of a graft device including a spine placed between layers of a fiber matrix, consistent with the present inventive concepts.

FIG. 2C is an end sectional view of a graft device including a spine placed outside a fiber matrix, consistent with the present inventive concepts.

FIGS. 3A-3F are end sectional views of a filament of a spine, consistent with the present inventive concepts.

FIG. 3G is a perspective view of a filament of a spine comprising multiple braided filaments, consistent with the present inventive concepts.

FIG. 4 is a perspective view of a spine including attachment elements and positioned on a tubular conduit, consistent with the present inventive concepts.

FIG. 4A is a magnified view of a portion of the spine of FIG. 4.

FIG. 5A is a perspective view of a two piece spine being applied to a tubular conduit, consistent with the present inventive concepts.

FIG. 5B is a perspective view of the two piece spine of FIG. 5A, after attachment around the tubular conduit, consistent with the present inventive concepts.

FIG. 6 is a schematic view of a system for producing a graft device, consistent with the present inventive concepts.

FIG. 7 is a side view of a system for producing a graft device with an electrospun fiber matrix, consistent with the present inventive concepts.

FIG. 8A is a perspective view of a fabrication tool for producing a spine for a graft device, consistent with the present inventive concepts.

FIG. 8B is a perspective view of the spine fabrication tool of FIG. 8A, with multiple pins inserted, consistent with the present inventive concepts.

FIG. 8C is a perspective view of the spine fabrication tool of FIGS. 8A and 8B, with a filament engaged about the tool, consistent with the present inventive concepts.

FIG. 8D is a perspective view of a spine fabricated by the tool of FIGS. 8A and 8B, consistent with the present inventive concepts.

FIG. 9 is a perspective view of a spine application tool with an engaged spine, consistent with the present inventive concepts.

FIGS. 9A-9F is a series of deployment steps including end views of the spine tool of FIG. 9 deploying a spine about a tubular conduit, consistent with the present inventive concepts.

FIG. 10 is a perspective view of a spine applied to a tubular conduit positioned on a mandrel, consistent with the present inventive concepts.

### DETAILED DESCRIPTION

The terminology used herein is for the purpose of describing particular embodiments and is not intended to be limiting of the inventive concepts. Furthermore, embodiments of the present inventive concepts may include several novel features, no single one of which is solely responsible for its desirable attributes or which is essential to practicing an inventive concept described herein. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

Provided herein are graft devices for implantation in a mammalian patient, such as to carry fluids (e.g. blood or other body fluid) from a first anatomical location to a second anatomical location. The graft devices include a tubular conduit, such as a harvested blood vessel segment or other harvested tissue, and a fiber matrix that surrounds the tubular conduit. The fiber matrix is typically applied with one or more of: an electrospinning device; a melt-spinning device; a melt-electrospinning device; a misting assembly; a sprayer; an electrosprayer; a fuse deposition device; a selective laser sintering device; a three-dimensional printer; or other fiber matrix delivery device. The fiber matrix delivery process can be performed in an operating room, such as when the tubular conduit is a harvested saphenous vein segment to be anastomosed between the aorta and a location on a diseased coronary artery distal to an occlusion. In these cardiovascular bypass procedures, end to side anastomotic connections are typically used to attach the graft device to the aorta and the diseased artery. Alternatively, a side to side anastomosis can be used, such as to attach an end of the graft device to multiple arteries in a serial fashion.

The graft devices can further include a spine, such as to prevent luminal narrowing, radial collapse, kinking and/or other undesired movement of the graft device (e.g. movement into an undesired geometric configuration), such as while implanting the graft device during a surgical procedure and/or at a time after implantation. The spine can be placed inside the tubular conduit, between the tubular conduit and the fiber matrix, between layers or within layers of the fiber matrix and/or outside the fiber matrix. The spine can comprise a biodegradable or bioerodible (hereinafter “biodegradable”) material or otherwise be configured to provide a temporary support to the graft device. Alternatively or additionally, the spine can comprise one or more portions including durable or otherwise non-biodegradable materials configured to remain intact for long periods of time when implanted, such as at least 6 months or at least 1 year.

Also provided herein are systems and methods for producing a graft device comprising a conduit, a surrounding fiber matrix and a spine. Systems typically include an electrospinning device and/or other fiber or fiber matrix delivering assembly. In some embodiments, the spine comprises a component that is applied, placed and/or inserted, such as by the fiber matrix delivery assembly (e.g. automatically or semi-automatically) or with a placement or insertion tool (e.g. manually).

The systems, methods, and devices of the present inventive concepts described herein can include an electrospun fiber matrix such as those disclosed in U.S. patent application Ser. No. 13/502,759, filed Sep. 10, 2012, the contents of which are incorporated herein by reference in its entirety. The graft devices described herein, as well as systems, tools and methods for producing graft devices, such as those disclosed in applicant's co-pending applications U.S. patent application Ser. No. 13/515,996, filed Jul. 11, 2012; U.S. patent application Ser. No. 13/811,206, filed Jan. 18, 2013; U.S. patent application Ser. No. 13/979,243, filed Aug. 6, 2013; U.S. patent application Ser. No. 13/984,249, filed Aug. 7, 2013; U.S. patent application Ser. No. 14/354,025, filed Apr. 24, 2014; and U.S. patent application Ser. No. 14/378,263, filed Aug. 12, 2014; the contents of each of which is incorporated herein by reference in its entirety.

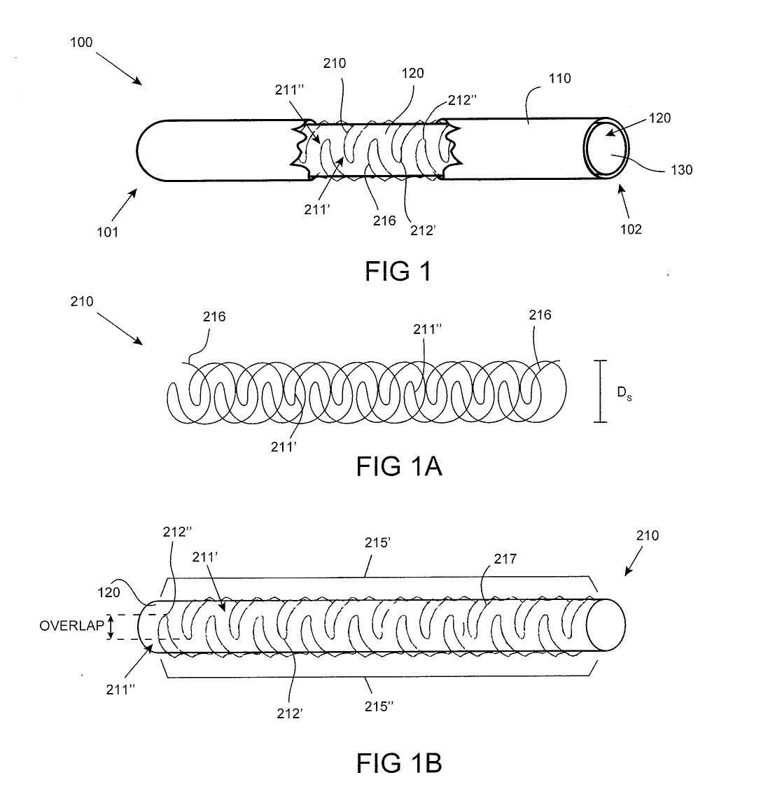

Referring now to FIG. 1, a perspective, partial cut-away view of a graft device including a spine is illustrated, consistent with the present inventive concepts. Graft includes tubular conduit 120, fiber matrix 110 and spine 210. In FIG. 1A, a side perspective view of spine 210 is illustrated. In FIG. 1B, spine 210 has been positioned about tubular conduit 120. In some embodiments, spine 210 has also been positioned about at least a portion (e.g. at least an inner layer) of fiber matrix 110.

Tubular conduit 120 is circumferentially surrounded by fiber matrix 110. Graft device 100 includes a first end 101 and a second end 102, and is preferably configured to be placed between a first body location and a second body location of a patient. Graft device 100 includes lumen 130 from first end 101 to second end 102, such as to carry blood or other fluid when graft device 100 is connected between two vessels, such as two blood vessels in a cardiovascular bypass procedure.

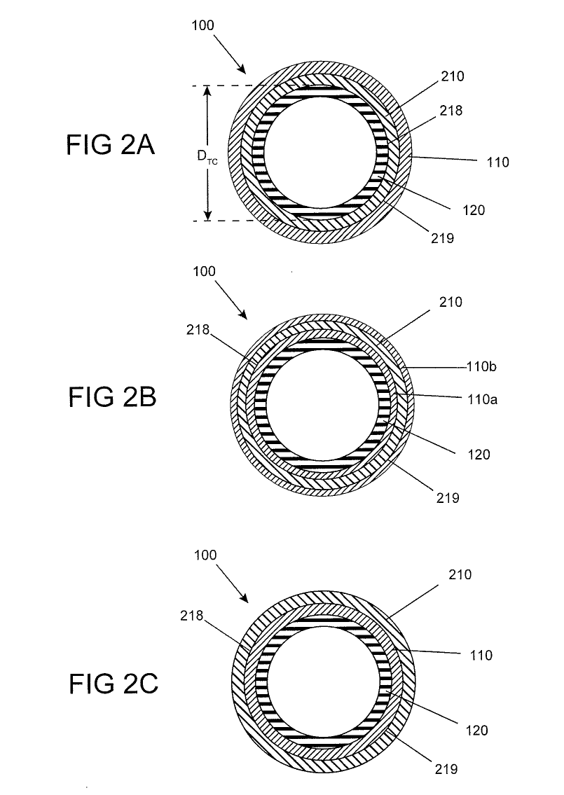

Graft device 100 further includes spine 210. Spine 210 is constructed and arranged to prevent graft device 100 from undergoing undesired motion such as kinking or other narrowing, such as during implantation procedure and/or while under stresses endured during its functional lifespan. In some embodiments, spine 210 surrounds conduit 120, positioned between conduit 120 and fiber matrix 110, as is shown FIG. 2A herebelow, where spine 210 comprises a diameter approximating the outer diameter of conduit 120. In some embodiments, spine 210, in whole or in part, can be between one or more layers of fiber matrix 110, such as is shown in FIG. 2B herebelow. In some embodiments, spine 210, in whole or in part, can surround fiber matrix 110, such as is shown in FIG. 2C. In some embodiments, spine 210 is positioned within conduit 120 (configuration not shown). In some embodiments, multiple spines 210 can be included, each surrounding tubular conduit 120, surrounding fiber matrix 110 and/or positioned between two or more layers of fiber matrix 110.

Spine 210 can be constructed and arranged to provide one or more functions selected from the group consisting of: minimizing undesirable conditions, such as buckling, conduit 120 deformation, luminal deformation, stasis, flows characterized by significant secondary components of velocity vectors such as vortical, recirculating or turbulent flows, luminal collapse, and/or thrombus formation; preserving laminar flow such as preserving laminar flow with minimal secondary components of velocity, such as blood flow through graft device 100, blood flow proximal to graft device 100 and/or blood flow distal to graft device 100; preventing bending and/or allowing proper bending of the graft device 100, such as bending that occurs during and/or after the implantation procedure; preventing accumulation of debris; preventing stress concentration on the tubular wall; maintaining a defined geometry in tubular conduit 120; preventing axial rotation about the length of tubular conduit 120; and combinations thereof. Spine 210 and fiber matrix 110 can comprise similar elastic moduli, such as to avoid dislocations and/or separations between the two components over time, such as when graft device 100 undergoes cyclic motion and/or strain.

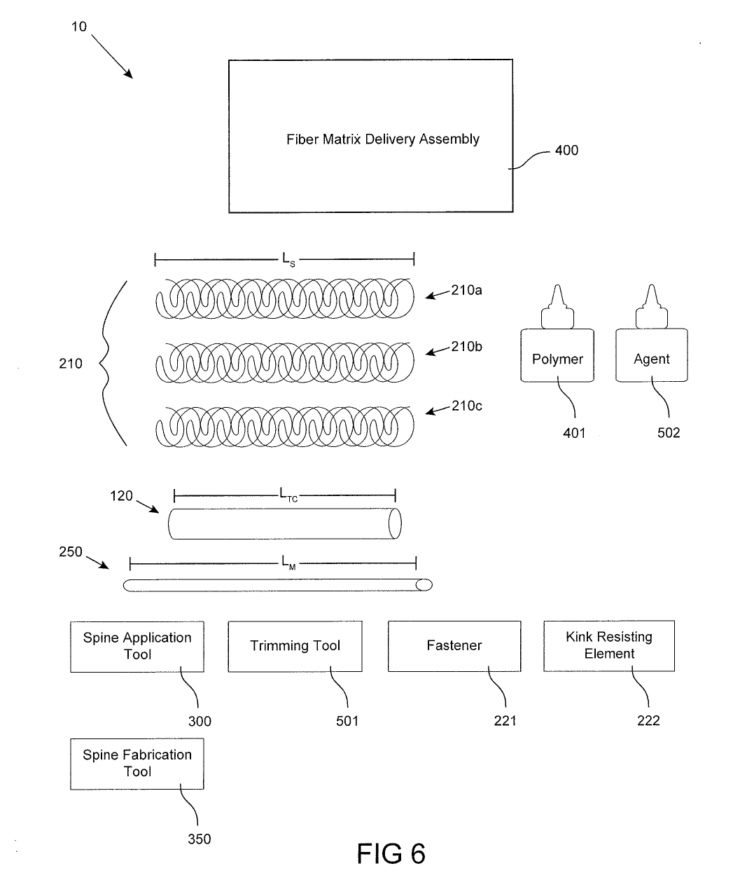

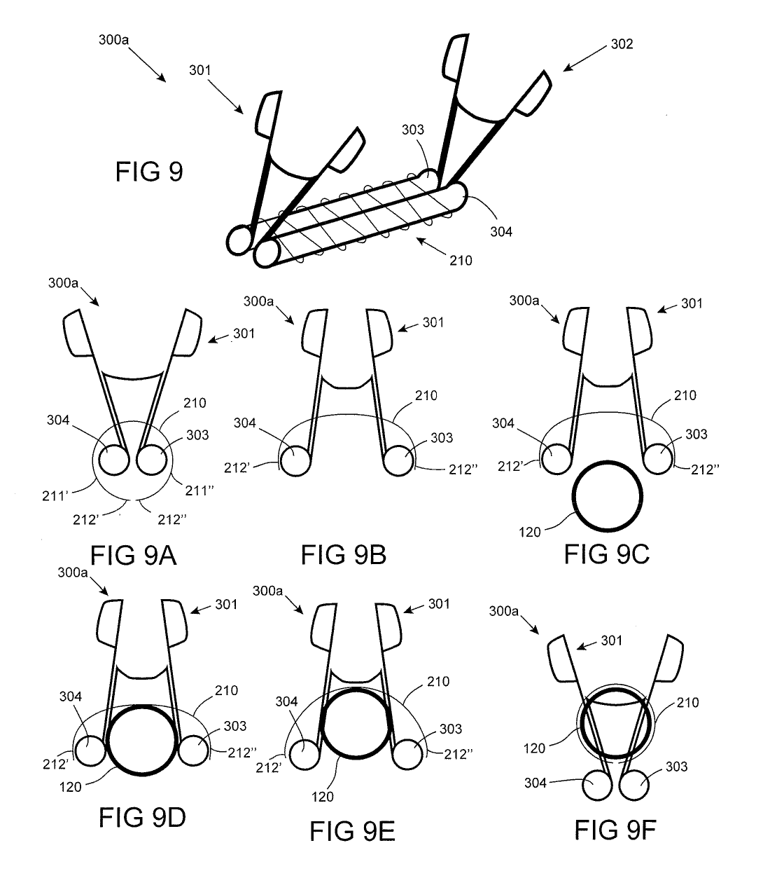

Spine 210 can be applied around conduit 120 prior to, during and/or after application of fiber matrix 110 to graft device 100. For example, spine 210 can be applied prior to application of fiber matrix 110 when spine 210 is positioned between conduit 120 and fiber matrix 110, as shown in FIG. 2A. Spine 210 can be applied during application of fiber matrix 110 when spine 210 is positioned between one or more layers of fiber matrix 110, as shown in FIG. 2B. Spine 210 can be applied after application of fiber matrix 110 when spine 210 is positioned outside of fiber matrix 110, as shown in FIG. 2C. Spine 210 can be applied about conduit 120 and/or at least a layer of fiber matrix 110 with one or more tools, such as tool 300 described in reference to FIG. 6, or tool 300a described in reference to FIG. 9 herebelow.

Spine 210 can include one or more portions that are resiliently biased, such as a resilient bias configured to provide a radial outward force at locations proximate ends 101 and/or 102, such as to provide a radial outward force to support or enhance the creation of an anastomosis as described herein. Spine 210 can include one or more portions that are malleable.

In some embodiments, spine 210 includes multiple curved projections 211′ and 211″, singly or collectively projections 211. Projections 211′ each include a tip portion 212′ and projections 211″ each include a tip portion 212″ (singly or collectively, tip portions 212). In some embodiments, each tip portion 212 can comprise a diameter between 0.020″ and 0.064″, such as a diameter approximating 0.042″. Projections 211 can each comprise a loop of a filament (e.g. a loop of a continuous filament), and projections 211′ and 211″ can be arranged in an interdigitating arrangement such as the alternating, interdigitating arrangement shown in FIGS. 1, 1A and 1B. In some embodiments, the interdigitating projections 211′ and 211″ can overlap (e.g. spine 210 covers more than 360° of conduit 120). In some embodiments, projections 211′ and 211″ are arranged with an overlap of at least 1.0 mm, at least 1.1 mm or at least 1.4 mm. A set of projections 211′ can comprise first support portion 215′, whose tip portions 212′ can be collectively deflected or otherwise rotated towards the top of the page. A set of projections 211″ can comprise a second support portion 215″, whose tip portions 212″ can be collectively deflected or otherwise rotated towards the bottom of the page. The rotations of first support portion 215′ and second support portion 215″ create an opening that allows spine 210 to approach and surround conduit 120 from the side (e.g. laterally engage conduit 120 and/or at least a layer of fiber matrix 110 already applied to conduit 120). Rotation of first support portion 215′ relative to second support portion 215″ and/or rotation of second support portion 215″ relative to first support portion 215′ can be performed with one or more spine application tools, such as tools 300 and 300a described herebelow.

Spine 210 can comprise at least three projections 211, such as at least six projections 211. In some embodiments, spine 210 includes at least two projections 211 for every 15 mm of length of spine 210, such as at least two projections 211 for every 7.5 mm of length of spine 210, or at least two projections 211 for every 2 mm of length of spine 210. In some embodiments, spine 210 comprises two projections 211 for each approximately 6.5 mm of length of spine 210.

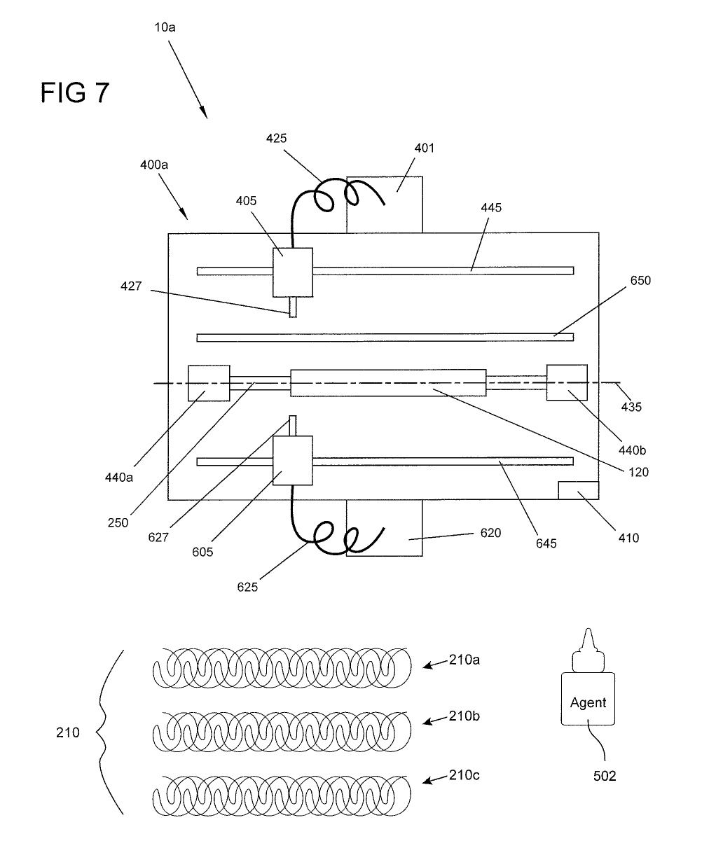

Spine 210 can comprise one or more continuous filaments 216, such as three or less continuous filaments, two or less continuous filaments, or a single continuous filament as shown in FIG. 1A. In some embodiments, spine 210 comprises a continuous filament 216 of at least 15″ long, or at least 30″ long such as when spine 210 comprises a length of approximately 3.5″. In some embodiments, filament 216 comprises a length (e.g. a continuous length or a sum of segments with a cumulative length) of approximately 65″ (e.g. to create a 4.0 mm diameter spine 210), or a length of approximately 75″ (e.g. to create a 4.7 mm diameter spine 210), or a length of approximately 85″ (e.g. to create a 5.5 mm diameter and/or 3.5″ long spine 210). Filament 216 can comprise a relatively continuous cross section, such as an extruded or molded filament with a relatively continuous cross section. Spine 210 can comprise a filament 216 including at least a portion with a cross section with a geometry selected from the group consisting of: elliptical; circular; oval; square; rectangular; trapezoidal; parallelogram-shaped; rhomboid-shaped; T-shaped; star-shaped; spiral-shaped; (e.g. a filament comprising a rolled sheet); and combinations of these, such as is shown in FIGS. 2A through 2E. Filament 216 can comprise a cross section with a major axis between approximately 0.2 mm and 1.5 mm in length, such as a circle or oval with a major axis less than or equal to 1.5 mm, less than or equal to 0.8 mm, or less than or equal to 0.6 mm, or between 0.4 mm and 0.5 mm. Filament 216 can comprise a cross section with a major axis greater than or equal to 0.1 mm, such as a major axis greater than or equal to 0.3 mm. In some embodiments, the major axis and/or cross sectional area of filament 216 is proportionally based to the diameter of spine 210 (e.g. a larger spine 210 diameter correlates to a larger filament 216 diameter, such as when a range of different diameter spine 210's are provided in a kit as described in reference to FIGS. 6 and 7 herebelow).

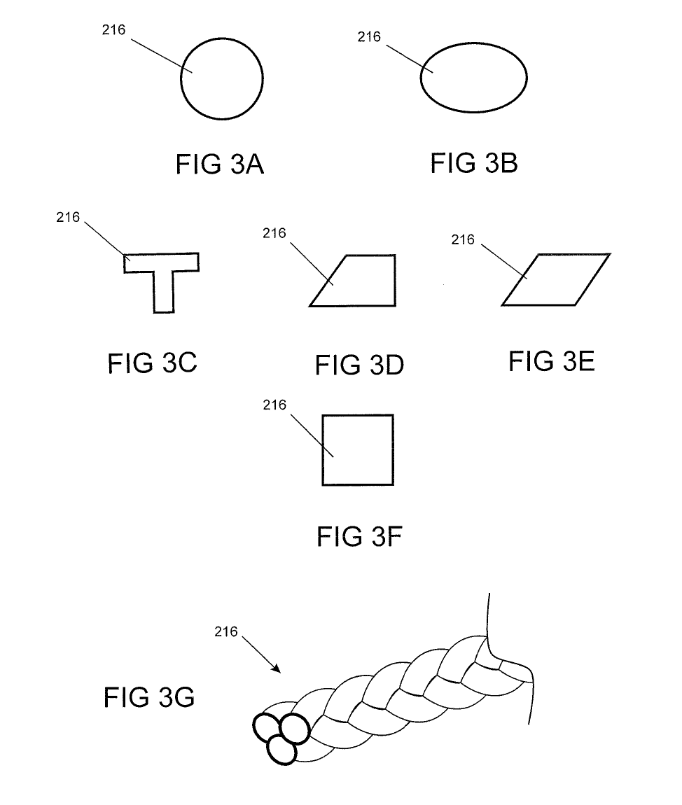

Filament 216 can be a single core or monofilament structure. Alternatively, filament 216 can comprise multiple filaments, such as a braided structure as shown in FIG. 3G. In some embodiments, filament 216 can comprise an injection molded component or a thermoset plastic component, such as when spine 210 comprises multiple projections 211 that are created at the same time as the creation of one or more filaments 216 (e.g. when filament 216 is created in a three dimensional shape).

Filament 216 can comprise an electrospun component, such as a component fabricated by the same electrospinning device used to create fiber matrix 110, such as when spine 210 and fiber matrix 110 comprise the same or similar materials.

Spine 210 can comprise a tubular structure, such as a full circumferential (e.g. at least) 360° or partial circumferential tubular structure. In some embodiments, spine 210 comprises an inner diameter D_{S} that approximates the outer diameter of tubular conduit 120, diameter D_{TC} as shown in FIG. 2A. In some embodiments, spine 210 comprises an inner diameter D_{S} that approximates the outer diameter of a partial layer of fiber matrix 110 covering tubular conduit 120. In some embodiments, spine 210 comprises an inner diameter D_{S} that approximates the outer diameter of a full layer of fiber matrix 110 covering tubular conduit 120. Spine 210 can comprise an inner diameter of at least 2 mm or an inner diameter of no more than 20 mm. Spine 210 can comprise a length between 2″ and 6″, such as a length between 3″ and 5″. In some embodiments, spine 210 comprises multiple tubular structures with lengths between 1″ and 4″, such as spines 210a, 210b and 210c described in reference to FIGS. 6 and 7 herebelow.

Spine 210 can comprise a material with a durometer between 52 D and 120 R, such as between 52 D and 85 D, such as between 52 D and 62 D. In some embodiments, spine 210 comprises a material with a durometer of approximately 55 D. Spine 210 can comprise one or more polymers, such as a polymer selected from the group consisting of: silicone; polyether block amide; polypropylene; nylon; polytetrafluoroethylene; polyethylene; ultra high molecular weight polyethylene; polycarbonates; polyolefins; polyurethanes; polyvinylchlorides; polyamides; polyimides; polyacrylates; polyphenolics; polystyrene; polycaprolactone; polylactic acid; polyglycolic acid; polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations of these. Spine 210 can comprise the same material as fiber matrix 110, such as when both comprise the same electrospun material.

Spine 210 can comprise at least one thermoplastic co-polymer. Spine 210 can comprise two or more materials, such as a first material and a second material harder than the first material. In some embodiments, Spine 210 can comprise relatively equal amounts of a harder material and a softer material. The softer material can comprise polydimethylsiloxane and a polyether-based polyurethane and the harder material can comprise aromatic methylene diphenyl isocyanate. Spine 210 can comprise one or more drugs or other agents, such as one or more agents constructed and arranged to be released over time.

In some embodiments, spine 210 comprises a metal material, such as a metal selected from the group consisting of: nickel titanium alloy; titanium alloy; titanium; stainless steel; tantalum; magnesium; cobalt-chromium alloy; gold; platinum; and combinations thereof. In some embodiments, spine 210 comprises a reinforced resin, such as a resin reinforced with carbon fiber and/or Kevlar. In some embodiments, at least a portion of spine 210 is biodegradable, such as when spine 210 comprises a biodegradable material such as a biodegradable metal or biodegradable polymer. In these embodiments, fiber matrix 110 can comprise a biodegradable material and/or a non-biodegradable material. In some embodiments, spine 210 does not comprise a biodegradable material. In these embodiments, fiber matrix 110 can comprise a biodegradable material and/or a non-biodegradable material.

Spine 210 can be configured to biodegrade over time such as to provide a temporary kink resistance or other function to device 100. In some embodiments, spine 210 can temporarily provide kink resistance to graft device 100 for a period of less than twenty-four hours. In some embodiments, spine 210 can provide kink resistance to graft device 100 for a period of less than one month. In some embodiments, spine 210 can provide kink resistance to graft device 100 for a period of less than six months. Numerous forms of biodegradable materials can be employed. Bolz et al. (U.S. patent Ser. No. 09/339,927) discloses a bioabsorbable implant which includes a combination of metal materials that can be an alloy or a local galvanic element. Metal alloys can consist of at least a first component which forms a protecting passivation coat and a second component configured to ensure sufficient corrosion of the alloy. The first component is at least one component selected from the group consisting of: magnesium, titanium, zirconium, niobium, tantalum, zinc and silicon, and the second component is at least one metal selected from the group consisting of: lithium, sodium, potassium, manganese, calcium and iron. Furst et al. (U.S. patent application Ser. No. 11/368,298) discloses an implantable device at least partially formed of a bioabsorbable metal alloy that includes a majority weight percent of magnesium and at least one metal selected from calcium, a rare earth metal, yttrium, zinc and/or zirconium. Doty et al. (U.S. patent application Ser. No. 11/744,977) discloses a bioabsorbable magnesium reinforced polymer stent that includes magnesium or magnesium alloys. Numerous biodegradable polymers can be used such as: polylactide, poylglycolide, polysaccharides, proteins, polyesters, polyhydroxyal kanoates, polyalkelene esters, polyamides, polycaprolactone, polyvinyl esters, polyamide esters, polyvinyl alcohols, polyanhydrides and their copolymers, modified derivatives of caprolactone polymers, polytrimethylene carbonate, polyacrylates, polyethylene glycol, hydrogels, photo-curable hydrogels, terminal diols, and combinations thereof. Dunn et al. (U.S. Pat. No. 4,655,777) discloses a medical implant including bioabsorbable fibers that reinforce a bioabsorbable polymer matrix.

Spine 210 can comprise a polymer constructed and arranged to change one or more properties upon exposure to an external stimuli. The polymer can comprise a polymer selected from the group consisting of: N-isopropylacrylamide (NIPAAm); a polaxamer (Pluronics); and combinations of these. The external stimuli can comprise a stimuli selected from the group consisting of: temperature; pH; light; magnetic field; electric field; exposure to a solvent; and combinations of these. The changed property can comprise a property selected from the group consisting of: hydrophobicity; a material property; an adhesive property; size; geometry; and combinations thereof. For instance, spine 210 can exhibit an increase of hydrophobicity when exposed to a stimuli such as an electromagnetic field, such as an electromagnetic field provided during an electrospinning process as described herein.

Spine 210 can comprise one or more coatings 217 as shown in FIG. 1B. Coating 217 can cover all or a portion of one or more filaments 216. Spine 210 can comprise an inner surface 218 and an outer surface 219 (each as described in reference to FIGS. 2A-C herebelow), and coating 217 can be positioned on inner surface 218, on outer surface 219, and/or on another surface of spine 210. Coating 217 can comprise an adhesive element or otherwise exhibit adhesive properties, such as a coating comprising a material selected from the group consisting of: fibrin gel; starch-based compound; mussel adhesive protein; and combinations of these. Coating 217 can be constructed and arranged to provide a function selected from the group consisting of: anti-thrombogenecity; anti-proliferation; anti-calcification; vasorelaxation; and combinations of these. Coating 217 can comprise a dehydrated gelatin, such as a dehydrated gelatin coating configured to hydrate to cause adherence of spine 210 to conduit 120. Coating 217 can comprise a hydrophilic and/or a hydrophobic coating. Coating 217 can comprise a radiopaque coating. In some embodiments, spine 210 comprises at least a portion that is radiopaque, such as when spine 210 comprises a radiopaque material such as barium sulfate.

Spine 210 can be constructed and arranged to be cut to length during the manufacturing process, such as at a time after application of at least a portion of fiber matrix 110. Spine 210 can be cut with one or more tools, such as trimming tool 501 described in reference to FIG. 6 herebelow.

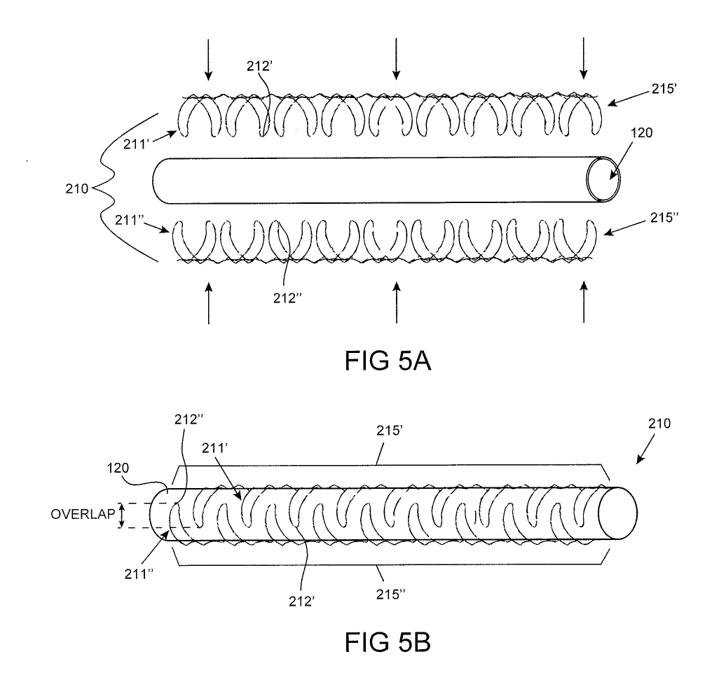

In some embodiments, spine 210 comprises a first portion and a separate (e.g. non-attached), second portion, such as a first support portion 215′ that is attachable to a second support portion 215″, as shown and described in reference to FIGS. 5A and 5B herebelow.

In some embodiments, one or more portions of the surface of spine 210 are modified. Spine 210 can be modified with one or more processes and/or surface modifying agents, such as by agent 502 and/or the surface modifying processes described in reference to FIG. 6 herebelow.

Tubular conduit 120 can comprise a varying circumferential shape, and fiber matrix 110 and/or spine 210 can be constructed and arranged to conform to the varying circumferential shape of conduit 120. Conduit 120 can comprise harvested tissue, such as a segment of a harvested vessel, such as a saphenous vein or other vein. In some embodiments, conduit 120 comprises tissue selected from the group consisting of: saphenous vein; vein; artery; urethra; intestine; esophagus; ureter; trachea; bronchi; duct; fallopian tube; and combinations of these. Alternatively or additionally, conduit 120 can comprise artificial material, such as a material selected from the group consisting of: polytetrafluoroethylene (PTFE); expanded PTFE (ePTFE); polyester; polyvinylidene fluoride/hexafluoropropylene (PVDF-HFP); silicone; polyethylene; polypropylene; polyester-based polymer; polyether-based polymer; thermoplastic rubber; and combinations of these.

Fiber matrix 110 can comprise one or more layers, such as a fiber matrix 110 with a thickness between 100 microns and 1000 microns, such as a thickness between 150 microns and 400 microns, or approximately 250 microns. In some embodiments, fiber matrix 110 comprises an inner layer and an outer layer, with spine 210 positioned therebetween, as described in reference to FIG. 2B herebelow. Fiber matrix 110 can comprise at least one polymer such as a polymer selected from the group consisting of: polyolefins; polyurethanes; polyvinylchlorides; polyamides; polyimides; polyacrylates; polyphenolics; polystyrene; polycaprolactone; polylactic acid; polyglycolic acid; and combinations of these. The polymer can be applied in combination with a solvent where the solvent is selected from the group consisting of: hexafluoroisopropanol; acetone; methyl ethyl ketone; benzene; toluene; xylene; dimethyleformamide; dimethylacetamide; propanol; ethanol; methanol; propylene glycol; ethylene glycol; trichloroethane; trichloroethylene; carbon tetrachloride; tetrahydrofuran; cyclohexone; cyclohexpropylene glycol; DMSO; tetrahydrofuran; chloroform; methylene chloride; and combinations of these. Fiber matrix 110 can comprise a thermoplastic co-polymer including two or more materials, such as a first material and a harder second material. Fiber matrix 110 can comprise one or more relatively durable (i.e. non-biodegradable) materials and/or one or more biodegradable materials, such as have been described hereabove. In some embodiments, fiber matrix 110 comprises a material selected from the group consisting of: polyglycerol sebacate; hyaluric acid; silk fibroin collagen; elastin; poly(p-dioxanone); poly(3-hydroxybutyrate); poly(3-hydroxyvalerate); poly(valcrolactone); poly(tartronic acid); poly(beta-malonic acid); poly(propylene fumarates); a polyanhydride; a tyrosine-derived polycarbonate; a polyorthoester; a degradable polyurethane; a polyphosphazene; and combinations of these.

Fiber matrix 110 can comprise an electrospun fiber matrix. In some embodiments, at least a portion of fiber matrix 110 is applied with a device selected from the group consisting of: an electrospinning device; a melt-spinning device; a melt-electrospinning device; a misting assembly; a sprayer; an electrosprayer; a three-dimensional printer; and combinations of these.

In some embodiments, device 100 comprises one or more fasteners configured to apply a retention force between at least two of tubular conduit 120, spine 210 and fiber matrix 110, such as fastener 221 described in reference to FIG. 6 herebelow. In some embodiments, device 100 comprises one or more kink resisting elements configured to prevent undesired movement of device 100, such as kink resisting element 222 described in reference to FIG. 6 herebelow.

In some embodiments, device 100 is produced using system 10 of FIG. 6 or system 10a of FIG. 7, each as described herebelow.

FIGS. 2A, 2B and 2C illustrate different configurations of placement of one or more spines 210 in a graft device 100, consistent with the present inventive concepts. In each configuration, tubular conduit 120, fiber matrix 110 and spine 210 can be constructed and arranged similar to the corresponding components described in reference to FIG. 1 hereabove.

Referring now to FIG. 2A, an end sectional view of a graft device including a spine placed between a tubular conduit and a fiber matrix is illustrated, consistent with the present inventive concepts. In some embodiments, referring to FIG. 2A, spine 210 has been placed between conduit 120 and fiber matrix 110, such as when spine 210 is applied to conduit 120 prior to application of fiber matrix 110. Conduit 120 comprises an outer diameter D_{TC}. The inner diameter of spine 210 (diameter D_{S} as shown in FIG. 1A), can approximate or otherwise be similar to outer diameter D_{TC}. In some embodiments, inner diameter D_{S} of spine 210 is slightly greater than outer diameter D_{TC} of conduit 120. Spine 210 comprises an inner surface 218 which contacts the outer surface of tubular conduit 120. Spine 210 further comprises an outer surface 219 which contacts the inner surface of fiber matrix 110. Inner surface 218, outer surface 219 and/or another surface of spine 210 can comprise a coating, such as coating 217 described in reference to FIG. 1B hereabove.

Referring now to FIG. 2B, an end sectional view of a graft device including a spine placed between layers of a fiber matrix is illustrated, consistent with the present inventive concepts. In some embodiments, referring to FIG. 2B, spine 210 has been placed between one or more inner layers of fiber, inner layer 110a, and one or more outer layers of fiber, outer layer 110b. In some embodiments, spine 210 can be applied (e.g. laterally applied) to conduit 120 after inner layer 110a has been applied by an electrospinning device or other fiber matrix delivery assembly, as described herein, such as by interrupting the delivery of fiber matrix 110 to apply spine 210 over the already deposited inner layer 110a. In some embodiments, inner layer 110a comprises a thickness approximately one-half the thickness of outer layer 110b. In some embodiments, inner layer 110a comprises a thickness of approximately between 62 and 83 microns. In some embodiments, inner layer 110a comprises between 1% and 99% of the total thickness of fiber matrix 110, such as between 25% and 60% of the total thickness, or approximately 33% of the total thickness of fiber matrix 110. Spine 210 comprises an inner surface 218 which contacts the outer surface of inner layer 110a. Spine 210 further comprises an outer surface 219 which contacts the inner surface of outer layer 110b. Inner surface 218, outer surface 219 and/or another surface of spine 210 can comprise a coating, such as coating 217 described in reference to FIG. 1B hereabove.

Referring now to FIG. 2C, an end sectional view of a graft device including a spine placed outside a fiber matrix is illustrated, consistent with the present inventive concepts. In some embodiments, for example as depicted in FIG. 2C, spine 210 has been placed outside of fiber matrix 110, such as after application of fiber matrix 110 to conduit 120. Spine 210 comprises an inner surface 218 which contacts the outer surface of fiber matrix 110. Spine 210 further comprises an outer surface 219 which comprises the outer surface of graft device 100. Inner surface 218, outer surface 219 and/or another surface of spine 210 can comprise a coating, such as coating 217 described in reference to FIG. 1B hereabove.

In some embodiments, two or more spines 210 are placed in multiple locations, such as two or more locations selected from the group consisting of: between conduit 120 and fiber matrix 110 (as shown in FIG. 2A); between inner layer 110a and outer layer 110b of fiber matrix 110 (as shown in FIG. 2B); outside of fiber matrix 110 (as shown in FIG. 2C); and inside of tubular conduit 120 (not shown but similar to placement of an intravascular stent).

Referring now to FIGS. 3A-3F, end sectional views of a filament of a spine are illustrated, consistent with the present inventive concepts. One or more filaments 216 of a spine 210 can comprise cross sections with one or more geometries, such as the geometries similar to the geometries illustrated in FIGS. 3A-3E. The geometries can be created during a process selected from: extrusion; molding such as injection molding; and combinations of these. For example, at least a portion of filament 216 can comprise a circular cross section, as shown in FIG. 3A. At least a portion of filament 216 can comprise an oval cross section, as shown in FIG. 3B. At least a portion of filament 216 can comprise a T-shaped cross section, as shown in FIG. 3C. At least a portion of filament 216 can comprise a trapezoidal cross section, as shown in FIG. 3D. At least a portion of filament 216 can comprise a rhomboid or other parallelogram-shaped cross section, as shown in FIG. 3E. At least a portion of filament 216 can comprise a square or other rectangular cross section, as shown in FIG. 3F.

Referring now to FIG. 3G, a perspective view of a filament of a spine comprising multiple braided filaments is illustrated, consistent with the present inventive concepts. In some embodiments, one or more filaments 216 of a spine 210 can comprise a braided construction, such as is illustrated in FIG. 3G. In some embodiments, the filament 216 can comprise a braid of multiple filaments each including one or more various cross sections, such as one or more of the cross sections illustrated in FIGS. 3A-3F.

The filaments 216 of FIGS. 3A-3G can comprise a geometric configuration, include one or more materials and/or otherwise be constructed and arranged as described in reference to filament 216 of FIG. 1 described hereabove.

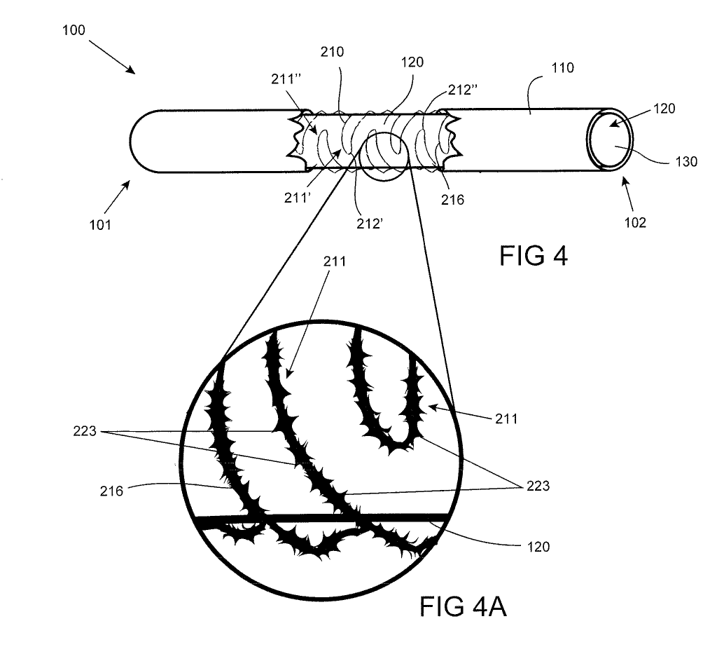

Referring now to FIG. 4, a perspective view of a spine including attachment elements and positioned on a tubular conduit is illustrated, consistent with the present inventive concepts. FIG. 4A is a magnified view of the spine of FIG. 4. Graft device 100 includes tubular conduit 120 and fiber matrix 110, such as are described in reference to FIG. 1 hereabove. Graft device 100 comprises first end 101, second end 102 with lumen 130 extending therebetween. Graft device 100 further includes spine 210, which has been placed to surround at least tubular conduit 120. In some embodiments, spine 210 further surrounds one or more layers of a fiber matrix, such as fiber matrix 110 described in reference to FIG. 2B hereabove. Spine 210 comprises one or more filaments 216 configured as multiple projections 211 with multiple tip portions 212, such as multiple interdigitating projections 211 as described herein. Spine 210 can comprise multiple barbs or other projections emanating from filament 216, retention members 223. Retention members 223 are constructed and arranged to provide a retention force between spine 210 and tubular conduit 120 and/or between spine 210 and fiber matrix 110. In some embodiments, retention members 223 are applied to filament 216 (e.g. with an adhesive). In some embodiments, spine 210 comprises one or more molded portions such that retention members 223 are created during the molding process of filament 216 and/or spine 210.

Referring now to FIG. 5A, a perspective view of a two-piece spine being applied to a tubular conduit is illustrated, consistent with the present inventive concepts. A spine 210 comprises a first support portion 215′ and a second support portion 215″ which comprise two separate components which are attachable to each other such as to surround tubular conduit 120. Spine 210 of FIG. 5A can be configured to partially circumferentially surround (e.g. less than) 360°, fully circumferentially surround (e.g. approximately 360°), or surround with overlap (e.g. more than 360°), conduit 120. First support portion 215′ comprises a first set of projections 211′ each with tip portion 212′. Second support portion 215″ comprises a second set of projections 211″ each with a tip portion 212″. First support portion 215′ and second support portion 215″ can be constructed and arranged similar to support portions 215′ and/or 215″ of FIG. 1 described hereabove. First support portion 215′ and/or second support portion 215″ can comprise an elongate member with a relatively continuous partial circumferential cross section, such as the approximately 180° cross section shown. In some embodiments, first support portion 215′ and/or second support portion 215″ can comprise similar or dissimilar cross sections, such as a cross section less than or equal to 180°. In FIG. 5B, first support portion 215′ and second support portion 215″ have been attached, such as by a clinician or other operator, such that first set of projections 211′ (each with tip portion 212′) interdigitate with second set of projections 211″ (each with tip portion 212″), and an assembled spine 210 surrounds conduit 120 with the overlap shown.

Referring now to FIG. 6, a schematic view of a system for producing a graft device is illustrated, consistent with the present inventive concepts. System 10 is constructed and arranged to produce a graft device 100 including a fiber matrix 110, such as is described herein. System 10 can be constructed and arranged to produce a graft device similar to graft device 100 of FIG. 1 described hereabove. System 10 comprises a fiber matrix delivery assembly 400, one or more spines, such as spine 210a, 210b and/or 210c (singly or collectively spine 210) as shown, and tubular conduit 120. Spine 210 and tubular conduit 120 can be provided in a sterile condition (e.g. when tubular conduit 120 comprises non-living tissue that has been sterilized), such as a spine 210 that has undergone a sterilization process known to those of skill in the art. System 10 can include a supply of material for producing a fiber matrix about conduit 120, such as polymer solution dispenser 401. Polymer solution dispenser 401 can comprise one or more polymers, solvents and/or other materials such as those described in reference to FIG. 1 hereabove. System 10 can include a tool for applying spine 210 about conduit 120, such as spine application tool 300 constructed and arranged to engage an inner and/or outer portion of spine 210 and subsequently cause spine 210 to radially expand to be placed (e.g. laterally placed) about conduit 120. In some embodiments, tool 300 can be constructed and arranged to maintain the geometry (e.g. shape or alignment) of one or more spines 210, such as to maintain the geometry of one or more spines 210 during shipping and/or storage.

In some embodiments, fiber matrix delivery assembly 400 comprises an assembly selected from the group consisting of: an electrospinning device; a melt-spinning device; a melt-electrospinning device; a misting assembly; a sprayer; an electrosprayer; a three-dimensional printer; or other fiber matrix delivery devices. In some embodiments, fiber matrix delivery assembly 400 comprises an electrospinning device, such as electrospinning device 400a described in reference to FIG. 7 herebelow. Fiber matrix delivery assembly 400 can be constructed and arranged to produce a fiber matrix of the present inventive concepts by delivering one or more polymers from polymer solution dispenser 401 to conduit 120 and/or spine 210.

Spines 210a, 210b and 210c can each comprise a length between 1″ and 6″, such as between 2″ and 6″ or between 3″ and 5″. In some embodiments, a single spine 210a, 210b or 210c is placed around conduit 120 to make a graft device of the present inventive concepts. In some embodiments, two or more spines 210, such as two or more of 210a, 210b or 210c are placed around conduit 120 in the process of producing a graft device of the present inventive concepts. A spine 210 can be selected for application around conduit 120 based on the size of conduit 120. In some embodiments, system 10 comprises a set of spines 210 including spines 210 with an inner diameter selected from the group consisting of: 4.0 mm; 4.7 mm; and 5.5 mm. In some embodiments, a spine 210 with an approximate inner diameter of 4.0 mm is chosen to surround a saphenous vein or other tubular conduit 120 with an approximate outer diameter of between 3.57 mm and 4.37 mm. In some embodiments, a spine 210 with an approximate inner diameter of 4.7 mm is chosen to surround a saphenous vein or other tubular conduit 120 with an approximate outer diameter of between 4.37 mm and 5.27 mm. In some embodiments, a spine 210 with an approximate inner diameter of 5.5 mm is chosen to surround a saphenous vein or other tubular conduit 120 with an approximate outer diameter of between 5.27 mm and 6.17 mm.

System 10 can include one or more mandrels 250 constructed and arranged to be slidingly inserted into tubular conduit 120 (e.g. an atraumatic insertion such as when tubular conduit 120 comprises a blood vessel). In some embodiments, mandrel 250 is constructed and arranged as described in applicant's co-pending applications U.S. patent application Ser. No. 13/984,249, filed Aug. 7, 2013; and U.S. patent application Ser. No. 14/364,989, filed Jun. 12, 2014; each of which is incorporated herein by reference in its entirety. After insertion into conduit 120, mandrel 250 can be inserted into fiber matrix delivery assembly 400, such as is described in reference to FIG. 7 herebelow. Material from polymer solution dispenser 401 is delivered by assembly 400 towards conduit 120 and/or spine 210 to create the fiber matrix 110, such as while mandrel 250 is being rotated.

In some embodiments, system 10 includes a spine application tool 300 constructed and arranged to apply (e.g. laterally apply) spine 210 to at least conduit 120 (e.g. conduit 120 and one or more inner layers of a fiber matrix 110). In some embodiments, spine application tool 300 comprises a robotic or otherwise at least partially automated tool, such as a robotic tool integrated into fiber matrix delivery assembly 400 such that spine 210 can be automatically or semi-automatically applied to surround conduit 120 (e.g. after mandrel 250 and conduit 120 have been operably engaged with fiber matrix delivery assembly 400). In some embodiments, tool 300 applies spine 210 to an inner layer of a fiber matrix 110 that has been applied to conduit 120, as has been described in reference to FIGS. 1 and 2B hereabove. In some embodiments, spine application tool 300 comprises manual tool, such as tool 300a described in reference to FIG. 9 herebelow. In some embodiments, one or more spines 210 can be supplied to a user already mounted or otherwise engaged with one or more tools 300.

System 10 can include one or more agents 502 for modifying the surface of spine 210, conduit 120 and/or a fiber matrix 110 applied by fiber matrix delivery assembly 400. In some embodiments, fiber matrix delivery assembly 400 delivers one or more agents 502 to one or more layers of the fiber matrix 110, such as an outer layer of the fiber matrix 110. Agent 502 can comprise a solvent configured to perform a surface modification, such as a solvent selected from the group consisting of: dimethylformamide; hexafluoroisopropanol; tetrahydrofuran; dimethyl sulfoxide; isopropyl alcohol; ethanol; and combinations thereof. In some embodiments, system 10 is constructed and arranged to perform a surface modification configured to enhance the adhesion of spine 210 to conduit 120 and/or the applied fiber matrix 110 comprising polymer from polymer solution dispenser 401. In some embodiments, system 10 is constructed and arranged to perform a surface modification to spine 210 to cause a modification of the surface energy of spine 210. In some embodiments, the surface of spine 210 is modified with a heated die comprising a textured or otherwise non-uniform surface. In some embodiments, fiber matrix delivery assembly 400 and/or another component of system 10 comprise a radiofrequency plasma glow discharge assembly constructed and arranged to perform a surface modification of spine 210, such as a process performed in the presence of a material selected from the group consisting of: hydrogen; nitrogen; ammonia; oxygen; carbon dioxide; C₂F₆; C₂F₄; C₃F₆; C₂H₄; C_{Z}H_{Z}; CH₄; and combinations of these.

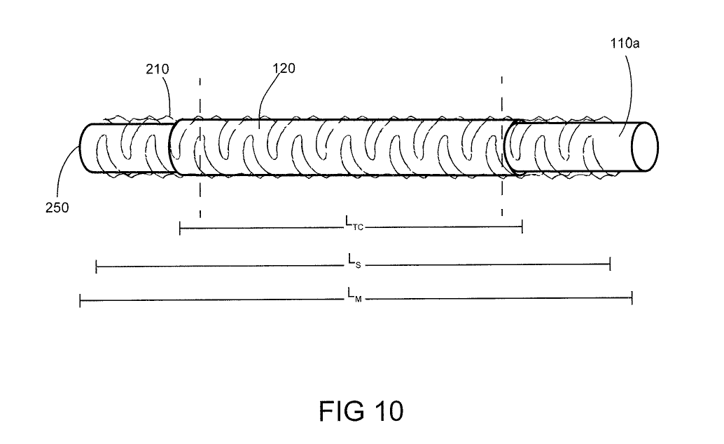

System 10 can include one or more tools for cutting or otherwise trimming one or more spines 210 to a particular length, such as trimming tool 501. Spine 210 and one or more portions of an applied fiber matrix 110 can be trimmed prior to, during or after application of one or more polymers from polymer solution dispenser 401 by fiber matrix delivery assembly 400. Trimming tool 501 can be a manual tool and/or an at least partially automated tool, such as a tool integrated into fiber matrix delivery assembly 400. In some embodiments, trimming tool 501 comprises one or more cutting tools such as a cutting tool selected from the group consisting of: scissors; scalpel; laser cutter; radiofrequency cutter; and combinations thereof. Tubular conduit 120 can comprise a harvested saphenous vein or other tissue or artificial conduit, mandrel 250 can be inserted into conduit 120, and subsequently tool 300 can be used to laterally apply a spine 210 to the assembly comprising mandrel 250 and conduit 120. In some embodiments, spine 210 comprises a length Lₛ that is longer than length L_{TC} of tubular conduit 120, and mandrel 250 comprises a length L_{M} that is longer than spine 210 length L_{S}, such as is described in reference to FIG. 10 herebelow. After application of the fiber matrix 110 to tubular conduit 120, spine 210 and mandrel 250, trimming tool 501 can be used to trim the fiber matrix 110 and/or spine 210 to a length approximating length L_{TC}. In some embodiments, spine 210 is at least 2 cm longer than conduit 120, such as at least 4 cm longer than conduit 120. Tubular conduit 120, spine 210 and the applied fiber matrix 110 comprising one or more polymers supplied by polymer solution dispenser 401 can be removed from mandrel 250 after trimmed by tool 501.

Fiber matrix delivery assembly 400 and/or another component of system 10 can be constructed and arranged to deliver a coating, such as a coating delivered to conduit 120, spine 210 and/or the fiber matrix 110 comprising one or more polymers from polymer solution dispenser 401. In some embodiments, the coating is similar to coating 217 described in reference to FIG. 1 hereabove.

System 10 can include one or more fasteners 221 configured to apply a retention force between at least two of tubular conduit 120, spine 210 and an applied fiber matrix 110 comprising one or more polymers from polymer solution dispenser 401. Fasteners 221 can comprise one or more elements selected from the group consisting of: adhesive; staple; clip; suture; barb; hook; and combinations of these. In some embodiments, fasteners 221 comprise at least 4 fasteners. In some embodiments, fasteners 221 are attached to and/or attachable to spine 210. Fasteners 221 can be applied to conduit 120 and/or spine 210 when conduit 120 and/or spine 210 are positioned about mandrel 250. Fasteners 221 can be positioned within 1 cm of one or both ends of conduit 120. In some embodiments, fasteners 221 comprise a material similar to the material of spine 210, such as the material of an interdigitating projection 211 of spine 210 as described herein.

System 10 can include one or more kink resisting elements 222 constructed and arranged to prevent kinking or other undesired movement of graft device 100 produced by system 10. In some embodiments, spine 210 comprises kink resisting element 222. In some embodiments, kink resisting element 222 is constructed and arranged similar to the kink resisting elements described in applicant's co-pending application U.S. patent application Ser. No. 14/378,263, filed Aug. 12, 2014, and incorporated herein by reference in its entirety.

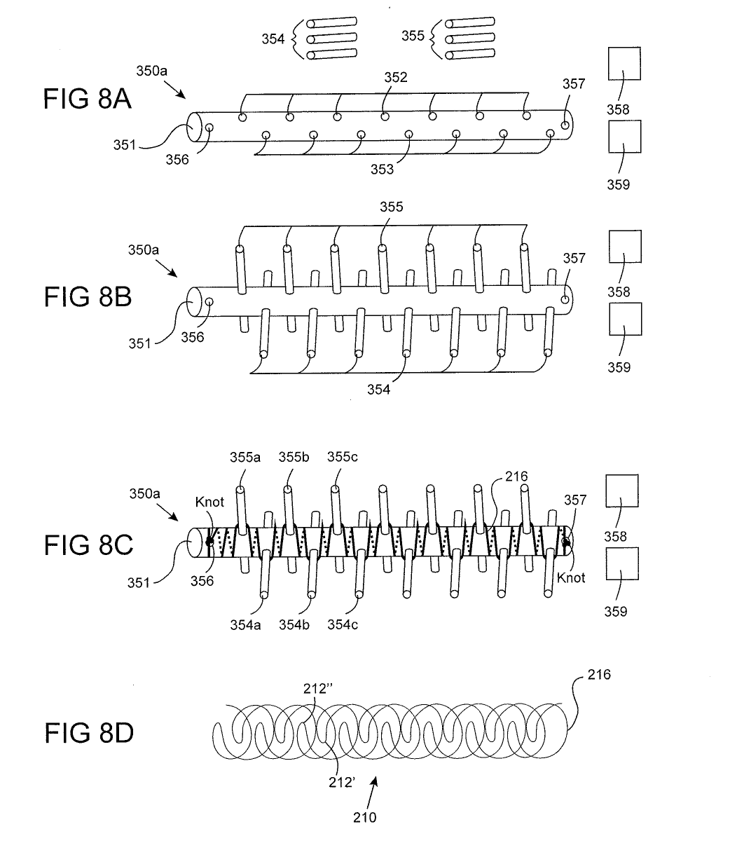

System 10 can include spine fabrication tool 350 which is constructed and arranged to produce one or more spines 210. Spine fabrication tool 350 can be constructed and arranged to resiliently bias spine 210, such as in a relatively linear or non-linear shape. In some embodiments, spine fabrication tool 350 is integral to fiber matrix delivery assembly 400. In these embodiments, fiber matrix delivery assembly 400 can create the spine 210 with an assembly selected from the group consisting of: an electrospinning device; a three-dimensional printer; a stereolithography device; a fuse deposition device; and combinations of these. In some embodiments, spine fabrication tool 350 is a separate device, such as spine fabrication tool 350a described in reference to FIGS. 8A-D herebelow. In some embodiments, spine fabrication tool 350 comprises one or more rods about which a filament is wrapped to create spine 210, such as two or more rods with different outer diameters used to produce two or more spines 210 with different inner diameters.

Referring now to FIG. 7, a side view of a system for producing a graft device with an electrospun fiber matrix is illustrated, consistent with the present inventive concepts. System 10a includes electrospinning device 400a and mandrel 250, where conduit 120 has been placed around mandrel 250. System 10a is constructed and arranged to produce a graft device 100 including a fiber matrix 110, such as is described herein. System 10a comprises one or more spines, such as spine 210a, 210b and/or 210c (singly or collectively spine 210) as shown. One or more spines 210 can be constructed and arranged as described in reference to one or more spines 210 described in reference to any of FIGS. 1-5b hereabove. Conduit 120 can include living tissue and/or artificial materials, as is described herein. Each end of mandrel 250 is inserted into a rotating drive, motor 440a and 440b, respectively, such that mandrel 250 can be rotated about center axis 435 during application of a fiber matrix (e.g. fiber matrix 110 described herein). Electrospinning device 400a can include one or more nozzle assemblies, and in the illustrated embodiment, electrospinning device 400a includes nozzle assembly 405, which includes one or more nozzles 427. Nozzle assembly 405 is fluidly attached to polymer solution dispenser 401 via delivery tube 425. Dispenser 401 comprises a solution of one or more polymers, solvents and/or other materials such as those described hereabove in reference to FIG. 1 hereabove. Nozzle assembly 405 is operably attached to a linear drive assembly 445 configured to translate nozzle assembly 405 in at least one direction.

Electrospinning device 400a can include one or more graft modification assemblies constructed and arranged to modify one or more components and/or one or more portions of graft device 100. In some embodiments, as illustrated, electrospinning device 400a includes modification assembly 605, which includes modifying element 627. Modification assembly 605 is operably attached to a linear drive assembly 645 configured to translate modification assembly 605 in at least one direction. Modification assembly 605 can be operably attached to supply 620 via delivery tube 625. System 10a can include one or more graft device 100 modifying agents, such as agent 502. Agent 502 can be constructed and arranged similar to agent 502 of FIG. 6 described hereabove. Supply 620 can comprise one or more of: a reservoir of one or more agents such as agent 502; a power supply such as a laser power supply; and a reservoir of compressed fluid. In some embodiments, modifying element 627 comprises a nozzle, such as a nozzle configured to deliver a fiber modifying agent and/or a graft modifying agent. For clarification, any reference to a “nozzle” and “nozzle assembly” in singular or plural form can include one or more nozzles, such as nozzle 427, and one or more assemblies, such as nozzle assemblies 405.

In some embodiments, modifying element 627 is configured to deliver a spine 210, such as a robotic assembly constructed and arranged to laterally deliver spine 210 about at least conduit 120. Alternatively or additionally, modifying element 627 can be configured to modify conduit 120, spine 210 and/or fiber matrix 110, such as to cause graft device 100 to be kink resistant or otherwise enhance the performance of the graft device 100 produced by system 10a. In these graft device 100 modifying embodiments, modifying element 627 can comprise a component selected from the group consisting of: a robotic device such as a robotic device configured to apply spine 210 to tubular conduit 120; a nozzle; an energy delivery element such as a laser delivery element such as a laser excimer diode or other last configured to trim one or more components of graft device 100; a fluid jet such as a water jet or air jet; a cutting element; a mechanical abrader; and combinations of these. Modification of fiber matrix 110 or other graft device 100 component can occur during the application of fiber matrix 110 and/or after fiber matrix 110 has been applied to conduit 120. Modification of one or more spines 210 can be performed prior to and/or after spine 210 has been applied to surround conduit 120.

Modifying element 627 can deliver an agent, such as agent 502. Modifying element 627 can comprise a fluid jet, such as a jet configured to deliver a gas or a liquid, such as air or a polymer solvent solution. In some embodiments, supply 620 can comprise a compressed air chamber configured to deliver compressed air through delivery tube 625 and modifying element 627, such as compressed air delivered during the application of fiber matrix 110 to conduit 120 by nozzle 427.

Modifying element 627 can comprise a laser delivery element, such as a laser diode. In this embodiment, supply 620 can comprise a power source and/or an electronic control module configured to power and control the pattern of laser energy delivered by modifying element 627. Laser energy can be delivered during and/or after the application of fiber matrix 110 to conduit 120. In some embodiments, modifying element 627 can be used to cut or otherwise trim fiber matrix 110 and/or a spine 210, similar to trimming tool 501 described in reference to FIG. 6 hereabove.

In some embodiments, modification assembly 605 of system 10a can be an additional component, separate from electrospinning device 400a, such as a handheld device configured to deliver spine 210, such as spine application tool 300 of FIG. 6 or tool 300a of FIG. 9 described herein. In some embodiments, modification assembly 605 comprises a handheld laser, such as a laser device which can be hand operated by an operator. Modification assembly 605 can be used to modify graft device 100 after removal from electrospinning device 400a, such as prior to and/or during an implantation procedure.

Laser or other modifications to fiber matrix 110 can cause portions of fiber matrix 110 to undergo physical changes, such as hardening, softening, melting, stiffening, creating a resilient bias, expanding, and/or contracting, and/or can also cause fiber matrix 110 to undergo chemical changes, such as forming a chemical bond with an adhesive layer between the outer surface of conduit 120 and fiber matrix 110. In some embodiments, modifying element 627 is alternatively or additionally configured to modify tubular conduit 120, such that tubular conduit 120 comprises a kink resisting or other performance enhancing element. Modifications to tubular conduit 120 can include but are not limited to a physical change to one or more portions of tubular conduit 120 selected from the group consisting of: hardening; softening; melting; stiffening; creating a resilient bias; expanding; contracting; and combinations of these. Modifications of tubular conduit 120 can cause tubular conduit 120 to undergo chemical changes, such as forming a chemical bond with an adhesive layer between an outer surface of conduit 120 and spine 210 and/or fiber matrix 110.

System 10a can include one or more patterning masks, such as a physical or chemical mask used to prevent fiber matrix 110 from covering one or more portions of conduit 120. In the illustrated embodiment, system 10a includes aperture plate 650. Aperture plate 650 can comprise a stencil-like pattern configured to prevent or reduce delivery of fiber to certain portions of the outer surface of conduit 120. In some embodiments, aperture plate 650 can comprise a stencil-like pattern that causes fiber matrix 110 to include a pattern of relief slots. Alternatively or additionally, aperture plate 650 can be configured to induce one or more changes to the electromagnetic (EM) field within electrospinning device 400a. These one or more changes to the EM field can be configured to cause variations in the delivered fiber pathway, resulting in a patterned fiber matrix 110. In some embodiments, mandrel 250 can have modified electrical characteristics, such as modified conductivity along its length, configured to modify the EM field to cause patterned fiber deposition.

In some embodiments, modification assembly 605 can comprise a masking agent delivery assembly. For example, supply 620 can store a masking solution configured to be delivered through delivery tube 625 to a nozzle-based modifying element 627. The masking solution can be delivered to conduit 120 before and/or during the electrospinning of fiber matrix 110. The masking solution can be configured to cause fiber matrix 110 to have a patterned deposition configured as a kink resisting element. A mask can be applied to conduit 120 prior to its insertion into electrospinning device 400a, such as by an operator applying a pre-manufactured covering or sleeve to conduit 120, or by painting or otherwise applying a substance, such as a chemical barrier, to mask conduit 120 from fiber matrix 110. In some embodiments, one or more masks are applied to conduit 120 to modify the EM field used in electrospinning, such as to reduce or prevent fiber deposition proximate the mask. The masks can comprise a biodegradable material, such as to dissolve over time after implantation of graft device 100. The masks can comprise a material which is configured to be washed off of or otherwise be removed from graft device 100 subsequent to the application of fiber matrix 110 to conduit 120.

In some embodiments, fiber matrix 110 can include an inner layer and an outer layer, where the inner layer can include an adhesive component and/or exhibit adhesive properties. The inner layer can be delivered separate from the outer layer, for example, delivered from a separate nozzle or at a separate time during the process. Selective adhesion between the inner and outer layers can be configured to provide kink resistance. Spine 210 can be placed between the inner and outer layers of fiber matrix 110, such as is described in reference to FIG. 2B hereabove.

In some embodiments, electrospinning device 400a can be configured to deliver fiber matrix 110 and/or an adhesive layer according to set parameters configured to produce a kink resistant element in and/or provide kink resisting properties to device 100. For example, an adhesive layer can be delivered to conduit 120 for a particular length of time, followed by delivery of a polymer solution for another particular length of time. Other typical application parameters include but are not limited to: amount of adhesive layer and/or polymer solution delivered; rate of adhesive layer and/or polymer solution delivered; nozzle distance to mandrel 250 and/or conduit 120; linear travel distance of a nozzle or a fiber modifying element along its respective drive assembly (for example, drive assembly 445 or 645); linear travel speed of a nozzle or a fiber modifying element along its respective drive assembly; compositions of the polymer solution and/or adhesive layer; concentrations of the polymer solution and/or adhesive layer; solvent compositions and/or concentrations; fiber matrix 110 inner and outer layer compositions and/or concentrations; spontaneous or sequential delivery of the polymer solution and the adhesive layer; voltage applied to the nozzle; voltage applied to the mandrel; viscosity of the polymer solution; temperature of the treatment environment; relative humidity of the treatment environment; airflow within the treatment environment; and combinations of these.

Nozzle 427 can be constructed of stainless steel. In some embodiments, nozzle 427 has a tubular construction with a length of approximately 1.5″, an inner diameter (ID) of approximately 0.047″ and an outer diameter (OD) of approximately 0.065″. Nozzle 427 can include an insulating coating, with the tip of nozzle 427 exposed (e.g. non-insulated), such as with an exposed length of approximately 1 cm. Nozzle 427 geometry and electrical potential voltages applied between nozzle 427 and mandrel 250 are chosen to control fiber generation. In a typical embodiment, fibers are produced with an average diameter between 1.0 μm and 20 μm, such as between 5 μm and 15 μm, or between 6 μm and 12 μm.

Mandrel 250 is positioned in a particular spaced relationship from nozzle assembly 405 and/or modification assembly 605, and nozzle 427 and/or modifying element 627, respectively. In the illustrated embodiment, mandrel 250 is positioned above and below assemblies 605 and 405, respectively. Alternatively, mandrel 250 can be positioned either above, below, to the right and/or or to the left of, assembly 405 and/or assembly 605. The distance between mandrel 250 and the tip of nozzle 427 and/or modifying element 627 can be less than 20 cm, or less than 15 cm. In some embodiments, the tip of nozzle 427 and/or modifying element 627 is approximately 12.5 cm from mandrel 250. In some embodiments, multiple nozzles 427 and/or multiple modifying elements 627, for example components of similar or dissimilar configurations, can be positioned in various orientations relative to mandrel 250. In some embodiments, the distance between nozzles 427 and/or modifying elements 627 and mandrel 250 varies along the length of mandrel 250, such as to create a varying pattern of fiber matrix 110 along conduit 120. In some embodiments, nozzle 427 and/or modifying element 627 distances from mandrel 250 can vary continuously during the electrospinning process and/or the distance can vary for one or more set periods of time during the process.

In some embodiments, an electrical potential is applied between nozzle 427 and one or both of conduit 120 and mandrel 250. The electrical potential can draw at least one fiber from nozzle assembly 405 to conduit 120. Conduit 120 can act as the substrate for the electrospinning process, collecting the fibers that are drawn from nozzle assembly 405 by the electrical potential. In some embodiments, mandrel 250 and/or conduit 120 has a lower voltage than nozzle 427 to create the desired electrical potential. For example, the voltage of mandrel 250 and/or conduit 120 can be a negative or zero voltage while the voltage of nozzle 427 can be a positive voltage. Mandrel 250 and/or conduit 120 can have a voltage of about −5 kV (e.g., −10 kV, −9 kV, −8 kV, −7 kV, −6 kV, −5 kV, −4.5 kV, −4 kV, −3.5 kV, −3.0 kV, −2.5 kV, −2 kV, −1.5 kV, −1 kV) and the nozzle 427 can have a voltage of about +15 kV (e.g., 2.5 kV, 5 kV, 7.5 kV, 12 kV, 13.5 kV, 15 kV, 20 kV). In some embodiments, the potential difference between nozzle 427 and mandrel 250 and/or conduit 120 can be from about 5 kV to about 30 kV. This potential difference draws fibers from nozzle 427 to conduit 120. In some embodiments, nozzle 427 is placed at a potential of +15 kV while mandrel 250 is placed at a potential of −5 kV. In some embodiments, mandrel 250 is a fluid mandrel, such as the fluid mandrel described in applicant's co-pending U.S. patent application Ser. No. 13/997,933, filed Aug. 8, 2013, which is incorporated herein by reference in its entirety.

In some embodiments, a polymer solution, stored in polymer solution dispenser 401, is delivered to nozzle assembly 405 through polymer solution delivery tube 425. The electrical potential between nozzle 427 and conduit 120 and/or mandrel 250 can draw the polymer solution through nozzle 427 of nozzle assembly 405. Electrostatic repulsion, caused by the fluid becoming charged from the electrical potential, counteracts the surface tension of a stream of the polymer solution at nozzle 427 of the nozzle assembly 405. After the stream of polymer solution is stretched to its critical point, one or more streams of polymer solution emerges from nozzle 427 of nozzle assembly 405, and/or at a location below nozzle assembly 405, and move toward the negatively charged conduit 120. Using a volatile solvent, the solution dries substantially during transit and the fiber is deposited on conduit 120.

Mandrel 250 is configured to rotate about an axis, such as axis 435, with nozzle 427 typically oriented orthogonal to axis 435. The rotation around axis 435 allows fiber matrix 110 to be deposited along all sides, or around the entire circumference of conduit 120. In some embodiments, two motors 440a and 440b are used to rotate mandrel 250. Alternatively, electrospinning device 400a can include a single motor configured to rotate mandrel 250. The rate of rotation of mandrel 250 can determine how the electrospun fibers are applied to one or more segments of conduit 120. For example, for a thicker portion of fiber matrix 110, the rotation rate can be slower than when a thinner portion of fiber matrix 110 is desired.

In addition to mandrel 250 rotating around axis 435, the nozzle assembly 405 can move, such as when driven by drive assembly 445 in a reciprocating or oscillating horizontal motion. Drive assemblies 445, as well as drive assembly 645 which operably attaches to modification assembly 605, can comprise a linear drive assembly, not shown, but typically a belt-driven drive assembly comprising two or more pulleys driven by one or more stepper motors. Additionally or alternatively, assemblies 405 and/or 605 can be constructed and arranged to rotate around axis 435, rotating means not shown. The length of drive assemblies 445 and/or 645 and the linear motion applied to assemblies 405 and 605, respectively, can vary based on the length of conduit 120 to which a fiber matrix 110 is delivered and/or a fiber matrix 110 modification is applied. For example, the supported linear motion of drive assemblies 445 and/or 645 can be about 10 cm to about 50 cm. Assemblies 405 and/or 605 can move along the entire length or specific portions of the length of conduit 120. In some embodiments, fiber and/or modification is applied to the entire length of conduit 120 plus an additional 5 cm (to mandrel 250) on either or both ends of conduit 120. In some embodiments, fiber(s) and/or modification is applied to the entire length of conduit 120 plus at least 1 cm beyond either or both ends of conduit 120.

Assemblies 405 and/or 605 can be controlled such that specific portions along the length of conduit 120 are reinforced with a greater amount of fiber matrix 110 as compared to other or remaining portions. Alternatively or additionally, assemblies 405 and/or 605 can be controlled such that specific portions of the length of conduit 120 include one or more kink resistant elements positioned at those one or more specific conduit 120 portions. In addition, conduit 120 can be rotating around axis 435 while assemblies 405 and/or 605 move, via drive assemblies 445 and/or 645, respectively, to position assemblies 405 and/or 605 at the particular portion of conduit 120 to which fiber is applied and/or modified. In a typical embodiment, assemblies 405 and/or 605 are translated back and forth at a velocity of approximately 200 mm/sec. Rotational speeds of mandrel 250 and translational speeds of assemblies 405 and/or 605 can be relatively constant, or can be varied during the process.

System 10a can also include a power supply, power supply 410 configured to provide the electric potentials to nozzle 427 and mandrel 250, as well as to supply power to other components of system 10a such as drive assemblies 445 and 645 and modification assembly 605. Power supply 410 can be connected, either directly or indirectly, to at least one of mandrel 250 and conduit 120. Power can be transferred from power supply 410 to each component by, for example, one or more wires.

System 10a can also include inlet and/or outlet ports, not shown, but typically configured to control the environment surrounding the environment surrounding mandrel 250. A port can be configured to be both an inlet port and an outlet port. System 10a can include a housing, also not shown, but typically attachable to electrospinning device 400a and defining a chamber surrounding assemblies 405 and/or 605 and/or mandrel 250, such that the ports can control a more limited (smaller) environment surrounding assemblies 405 and/or 605 and/or mandrel 250. Additionally or alternatively, the ports can be used to introduce or remove one or more gases, introduce or remove humidity, control temperature, control sterility, provide other environmental controls, and combinations of these.

Referring now to FIG. 8A-8D, perspective views of a fabrication tool for producing a spine for a graft device is illustrated, consistent with the present inventive concepts. Spine fabrication tool 350a comprises rod 351. Rod 351 can comprise a metal rod, such as a stainless steel rod. Rod 351 can include a first set of holes 352 whose central axis lies in a first plane and pass from one side of rod 351 to a relatively opposite side of rod 351. Rod 351 includes a second set of holes 353 whose central axis lies in a second plane and pass from one side of rod 351 to a relatively opposite side of rod 351. Holes 352 may lie in a plane that is relatively orthogonal to the planes in which holes 353 lie, as shown in FIG. 8A. Rod 351 can include a hole 356 located proximate one end of rod 351, and a hole 357 located proximate the opposite end of rod 351. Tool 350a can comprise pins, such as pins 354 and 355. Pins 354 and 355 can be similar pins or dissimilar pins. Pins 354 are sized to be slidingly received by holes 353 and pins 355 are sized to be slidingly received by holes 352. Tool 350a can comprise a heater 358, such as an oven or heat gun used in a shape-forming process. Tool 350a can comprise an agent delivery device 359, such as a delivery device constructed and arranged to deliver a shape-forming agent such as a solvent or other chemical, or a cleaning agent such as isopropyl alcohol or fluid vibrated with ultrasonic waves.

In FIG. 8B, Pins 355 have been inserted into holes 352, and pins 354 have been inserted into holes 353 as shown. Pins 354 and 355 can comprise metal pins, such as stainless steel pins with a diameter less than 0.1″, such as pins with a diameter less than 0.050″ or a diameter of approximately 0.042″. Pins 354 and 355 can be inserted into rod 351 such that an equal length of each pin extends from opposite sides of rod 351.

Referring now to FIG. 8C, one end of a filament 216 has been attached to hole 356 near one end of rod 351, such as with a knot as shown. Filament 216 is wrapped around rod 351 in a first direction, proximate hole 356, such as with one or more circumferential turns about rod 351 (e.g. approximately two 360° turns as shown). Filament 216 is then wrapped around pin 355a (e.g. wrapped approximately 180° in a clockwise direction around pin 355a as shown on the page). Filament 216 is then wrapped around rod 351 (e.g. wrapped more than 360° in a second direction and passing between pin 355a and pin 354a as shown). Filament 216 is then wrapped around pin 354a (wrapped approximately 180° in a counter-clockwise direction around pin 354a as shown). Similar wrapping of filament 216 continues around pins 355b, 354b, 355c, 354c and so on in the interdigitating pattern shown in FIG. 8C. An approximate 180° wrapping of filament 216 around each pin 354 or 355 creates a tip portion 212 (e.g. tip portion 212′ or 212″) of each interdigitating projection 211 of spine 210 (as shown in FIG. 8D). The other end of filament 216 has been attached to hole 357, such as with a knot as shown. Tension can be applied to filament 216 during the wrapping process.

Spine fabrication tool 350a, including an attached (e.g. wound) filament 216 (as shown in FIG. 8C), may undergo one or more shape-forming processes to cause filament 216 to be biased (e.g. resiliently biased) in a tubular shape with interdigitating projections, such as via a heat setting process using heater 358, as is described in more detail herebelow. Alternatively or additionally, tool 350a and an attached filament 216 may be exposed to one or more solvents or other chemicals to resiliently bias filament 216, such as via agent delivery device 359. After one or more shape-biasing processes, pins 354 and 355 can be removed from rod 351 (e.g. pushed out of holes 353 and 352 respectively), and the processed filament 216 can be laterally and/or axially removed from rod 351. In some embodiments, filament 216 is cut proximate the knots at each end prior to removal of pins 354 and 355 and/or removal of filament 216 from rod 351, such as with trimming tool 501 of FIG. 6 or other cutting device. In some embodiments, filament 216 is cleaned prior to and/or after removal from tool 350a, such as with a cleaner delivered by agent delivery device 359. The processed filament 216 produced by tool 350a and one or more shape-biasing processes comprises spine 210 of the present inventive concepts, such as is shown in FIG. 8D.

Filament 216 can comprise an extrusion, such as an extruded polymer supplied on a spool. Filament 216 can be cleaned prior to application to tool 350a, such as a cleaning with isopropyl alcohol delivered by agent delivery device 359. Filament 216 may comprise various cross sectional geometries, such as are described hereabove in reference to FIGS. 2A-2G. In some embodiments, filament 216 comprises a cross section with a diameter of approximately 0.4 mm for creating a spine 210 with a diameter of approximately 4.0 mm. In some embodiments, filament 216 comprises a cross section with a diameter of approximately 0.5 mm for creating a spine 210 with a diameter between 4.7 mm and 5.5 mm.

In some embodiments, spine fabrication tool 350a is cleaned prior to application of filament 216 to tool 350a, such as a cleaning performed with an agent delivery device 359 comprising an ultrasonic cleaner. In some embodiments, tool 350a comprises multiple rods 351 with different outer diameters, such as to create spines 210 with different inner diameters. In some embodiments, one or more rods 351 comprise a diameter configured to create a spine 210 with a diameter of approximately 4.0 mm, 4.7 mm and/or 5.5 mm, such as a rod 351 with an approximate diameter of 4.0 mm, 4.7 mm and/or 5.5 mm, respectively.

In some embodiments, spine fabrication tool 350a with an attached filament 216 undergoes a thermosetting process to bias (e.g. resiliently bias) filament 216 in a proposed geometry, such as a thermosetting process performed using heater 358. The thermosetting process can include an exposure to an elevated temperature for a time period, such as a temperature of at least 100° C. for at least 10 minutes. After temperature exposure, surface modification process may be performed. In some embodiments, the surface modification process comprises agent delivery device 359 applying an agent such as dimethylformamide (DMF) to filament 216 (e.g. when still surrounding tool 350a). After exposure to the agent, a drying or secondary temperature exposure may occur, such as an exposure to approximately at least 100° C. for at least 10 minutes using heater 358.

While rod 351 is shown as a relatively linear tube, such that spine 210 will be biased in a relatively linear geometry, rod 351 can include one or more non-linear portions, such as a curved rod 351 constructed and arranged to resiliently bias spine 210 in a curved configuration such that the graft device 100 produced with spine 210 comprises a curved portion (e.g. to improve flow dynamics).

Referring now to FIG. 9, a perspective view of a spine application tool with an engaged spine is illustrated, consistent with the present inventive concepts. A spine 210 is shown engaged with a spine application tool 300a. Spine application tool 300a comprises an actuator assembly, including first handle 301 and a second handle 302. Handles 301 and 302 can comprise a scissor-like construction as shown. Spine 210 has been wrapped around force applying elements, elongate members 303 and 304 of tool 300a. Ends of elongate members 303 and 304 are attached to ends of handles 301 and 302. Elongate member 303 and 304 can each comprise an element selected from the group consisting of: tube; rod; plate; and combinations thereof. Handles 301 and 302 are each shown in an un-activated condition, such that elongate members 303 and 304 are in close proximity to each other. Activation of handles 301 and 302 causes elongate members 303 and 304 to separate, causing radial expansion of spine 210, as described in reference to FIGS. 9A-9F herebelow. In some embodiments, tool 300a comprises a locking mechanism, not shown but such as is included in interlocking scissor-handle tools such as locking forceps.

Referring now to FIGS. 9A through 9F, a series of deployment steps including end views of the spine tool of FIG. 9 deploying a spine about a tubular conduit is illustrated, consistent with the present inventive concepts. In FIG. 9A, spine 210 has been engaged with spline application tool 300a, such as by wrapping spine 210 around both of elongate members 303 and 304 as shown. Handles 301 and 302 (handle 301 shown, handle 302 positioned directly behind handle 301 on the page) are in the un-activated condition of FIG. 9 (i.e. each scissor handle finger holes apart). Spine 210 comprises a series of radial projections 211′ and 211″, whose tip portions 212′ and 212″, respectively, are in relative proximity to one another with tool 300a in the un-activated condition. In some embodiments, tip portions 212′ and 212″ overlap, as described herein. While tool 300a is shown as engaging an inner surface of spine 210, tool 300a can be constructed and arranged to engage an outer portion of spine 210 and/or to engage inner and/or outer surfaces of projections 211, such as to apply a force to cause spine 210 to radially expand as shown in FIG. 9B. In FIG. 9B, both handles 301 and 302 have been activated (handle 302 shown with the finger holes brought toward each other), such that elongate members 303 and 304 have moved apart, applying a force that causes radial expansion of spine 210 and a resultant separation between tip portions 212′ and 212″ (e.g. an opening is created between tip portions 212′ and 212″).

In FIG. 9C, handles 301 and 302 remain in the activated state (finger holes brought toward each other). Tool 300a and spine 210 laterally approach a tubular conduit 120 of the present inventive concepts. In FIG. 9D, handles 301 and 302 remain in the activated state, and tool 300a and spine 210 have advanced through the opening between tip portions 212′ and 212″ such that spine 210 partially surrounds conduit 120. In FIG. 9E, handles 301 and 302 remain in the activated state, and tool 300a and spine 210 have further advanced through the opening in between tip portions 212′ and 212″ such that spine 210 more fully surround conduit 120. In FIG. 9F, handles 301 and 302 have been transitioned to the un-activated state (finger holes brought away from each other), allowing radial compression of spine 210 (e.g. due to the resilient bias of spine 210), such that spine 210 at least partially surrounds and is in at least partial contact with conduit 120. Tool 300a has been advanced toward the bottom of the page, disengaging spine 210 from elongate members 303 and 304, after which tool 300a can be translated to move away from conduit 120 and spine 210.

In some embodiments, the application process of steps 9A-9F is performed when tubular conduit 120 is positioned on a mandrel and engaged with a fiber matrix delivery assembly such as fiber matrix delivery assembly 400 or electrospinning device 400a of FIG. 6 or 7, respectively, described hereabove. The spine 210 can be applied to conduit 120 after one or more layers (e.g. one or more inner layers as shown in FIG. 2B) of a fiber matrix 110 have been applied to conduit 120, as described hereabove.

Referring now to FIG. 10, a perspective view of a spine applied to a tubular conduit positioned on a mandrel is illustrated, consistent with the present inventive concepts. Tubular conduit 120 has been placed over mandrel 250, and spine 210 has been placed over tubular conduit 120. Conduit 120, mandrel 250 and spine 210 may be constructed and arranged as described hereabove. In some embodiments, one or more layers of a fiber matrix 110, such as inner layer 110a shown, have been electrospun or otherwise applied to conduit 120 prior to application of spine 210. Spine 210 may comprise a length greater than the length of conduit 120, such as a length at least 1 cm longer, at least 2 cm longer or at least 4 cm longer. In a subsequent step, a trimming process, such as a trimming process using trimming tool 501 described in reference to FIG. 6 hereabove, can be used to trim spine 210 and/or an applied fiber matrix 110 including inner layer 110a to a length similar to the length of conduit 120. In some embodiments, the length of conduit 120 is reduced at a similar time to the reduction in length of spine 210 and the applied fiber matrix 110.

While the graft devices of the present invention have been described in detail as including a fiber matrix and a spine, other types of coverings can be used, such as a one or more fibrous or non-fibrous conformal structures circumferentially surrounding a tubular conduit. The conformal structures can be modified to include the spine, and/or a separate spine can be included in the graft device.

While the preferred embodiments of the systems, methods and devices have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it can be possible, or even expedient in certain circumstances, to change the order in which some steps are performed, and it is intended that the particular steps of the method or procedure claim set forth herebelow not be construed as being order-specific unless such order specificity is expressly stated in the claim.

1. A graft device for a mammalian patient, comprising:
a tubular conduit;
a fiber matrix surrounding the tubular conduit; and
a spine comprising a first support portion and a second support portion, wherein at least one of the first support portion or the second support portion is constructed and arranged to rotate relative to the other to receive the tubular conduit.

2. A graft device for a mammalian patient, comprising:
a tubular conduit;
a fiber matrix surrounding the tubular conduit; and,
a spine comprising a first support portion comprising a first set of projections, and a second support portion comprising a second set of projections, wherein the first set of projections interdigitate with the second set of projections.

3. The device of any preceding device claim wherein the first support portion and the second support portion are constructed and arranged to rotate relative to each other to create an opening to receive the tubular conduit.

4. The device of any preceding device claim wherein the first support portion comprises a first set of projections and the second support portion comprises a second set of projections that interdigitate with the first set of projections.

5. The device of any preceding device claim wherein the spine is constructed and arranged to provide one or more functions selected from the group consisting of: minimizing undesirable conditions, such as buckling, conduit deformation, luminal deformation, stasis, flows characterized by significant secondary components of velocity vectors such as vortical, recirculating or turbulent flows, luminal collapse, and/or thrombus formation; preserving laminar flow such as preserving laminar flow with minimal secondary components of velocity, such as blood flow through the graft device, blood flow proximal to the graft device and/or blood flow distal to the graft device; preventing bending and/or allowing proper bending of the graft device, such as bending that occurs during and/or after the implantation procedure; preventing accumulation of debris; preventing stress concentration on the tubular wall; maintaining a defined geometry in the tubular conduit: preventing axial rotation about the length of the tubular conduit; and combinations thereof.

6. The device of any previous device claim wherein the spine is positioned between the tubular conduit and the fiber matrix.

7. The device of any previous device claim wherein the fiber matrix comprises an outer surface and wherein the spine is positioned on the fiber matrix outer surface.

8. The device of any preceding device claim wherein the fiber matrix comprises a thickness between 100 microns and 1000 microns.

9. The device of claim 8 wherein the fiber matrix comprises a thickness between 150 microns and 400 microns.

10. The device of claim 9 wherein the fiber matrix comprises a thickness of approximately 250 microns.

11. The device of any preceding device claim wherein the fiber matrix comprises an inner layer and an outer layer, and wherein the spine is positioned between the fiber matrix inner layer and outer layer.

12. The device of claim 11 wherein the fiber matrix comprises a first thickness and the inner layer comprises a second thickness approximately between 1% and 99% of the first thickness.

13. The device of claim 12 wherein second thickness comprises a thickness approximately between 25% and 60% of the first thickness.

14. The device of claim 13 wherein the second thickness comprises a thickness of approximately 33% of the first thickness.

15. The device of claim 11 wherein the inner layer comprises a layer of between approximately 62 microns and 83 microns in thickness.

16. The device of any preceding device claim wherein the fiber matrix comprises a first elastic modulus and the spine comprises a second elastic modulus similar to the first elastic modulus.

17. The device of any previous device claim wherein the spine is constructed and arranged to be positioned in the graft device prior to application of the fiber matrix to the graft device.

18. The device of any previous device claim wherein the spine is constructed and arranged to be positioned in the graft device during application of the fiber matrix to the graft device.

19. The device of any previous device claim wherein the spine is constructed and arranged to be positioned on the graft device after application of the fiber matrix to the graft device.

20. The device of claim 1 wherein the spine is constructed and arranged to be applied to the tubular conduit with a tool.

21.-274. (canceled)
